(19) United States
(12) Patent Application Publication
(10) Pub. No.: US 2023/0357708 A1
(43) Pub. Date: Nov. 9, 2023

(54) BIOCATALYTIC PRODUCTION OF PARA-HYDROXYBENZOIC ACID FROM METHANOL AND METHANE

(71) Applicant: The Board of Trustees of the Leland Stanford Junior University, Stanford, CA (US)

(72) Inventor: Nils AVERESCH, Stanford, CA (US)

(73) Assignee: The Board of Trustees of the Leland Stanford Junior University, Stanford, CA (US)

(21) Appl. No.: 18/246,966

(22) PCT Filed: Sep. 29, 2021
(86) PCT No.: PCT/US2021/052676

§ 371 Date: Mar. 28, 2023

Related U.S. Application Data
(60) Provisional application No. 63/085,567, filed on Sep. 30, 2020.

(51) Int. Cl. C12N 1/20; C12N 15/52; C12P 7/42; C12N 15/74; C12N 9/10; C12N 9/88; C12N 9/12; C12N 9/90

(52) U.S. Cl. CPC C12N 1/205; C12N 15/52; C12P 7/42; C12N 15/74; C12N 9/1085; C12N 9/88; C12N 9/1205; C12N 9/90; C12Y 205/01054; C12Y 504/04002; C12Y 402/0101; C12Y 401/0304; C12R 2001/26

(57) ABSTRACT

A method of producing para-hydroxybenzoic acid (pHBA) or a derivative thereof includes culturing the recombinant microorganism in a fermentation broth, wherein said recombinant microorganism comprising a genetically engineered pathway expressing at least one nucleic acid sequence encoding a polypeptide selected from: an exogenous chorismate pyruvate lyase of EC 5.4.4.2 or EC 4.1.3.40; an exogenous 3-deoxy-D-arabino-heptulosonate-7-phosphate (DAHP) synthase of EC 4.1.2.15, or EC 2.5.1.54; an exogenous shikimate kinase of EC 2.7.1.71; or an exogenous 3-dehydroquinate dehydratase (DHQ) of EC 4.2.1.10; adding a carbon source to the fermentation broth; and isolating the pHBA from the fermentation broth.

### CROSS-REFERENCE TO RELATED APPLICATIONS

This application claims the benefit of priority to U.S. Provisional Patent Application No. 63/085,567 filed Sep. 30, 2020, which is hereby incorporated by reference, in its entirety for any and all purposes.

### STATEMENT REGARDING FEDERALLY SPONSORED RESEARCH OR DEVELOPMENT

This invention was made with Government support under contract NNX17AJ31G awarded by NASA. The Government has certain rights in the invention.

### TECHNICAL FIELD

The present disclosure relates generally to biocatalytics, and more particularly to a biocatalytic production of para-hydroxybenzoic acid from C1 substrates using genetically engineered microorganisms.

### BACKGROUND

The following description of the background of the present technology is provided simply as an aid in understanding the present technology and is not admitted to describe or constitute prior art to the present technology.

Aromatic chemicals are very important building blocks for the fiber, coating, resin, and packaging industries as well as precursors for the pharmaceutical and cosmetic industries. Despite the fact that biology potentially offers greener and more sustainable alternatives, aromatics are still almost exclusively derived from fossil fuels.

The worldwide push to move toward a more sustainable society not only includes the goal to move from fossil fuel dependency toward renewable feedstocks but also aims to maintain and increase standards of living by facilitating the access to pharmaceuticals and securing the availability of foodstuff.

para-hydroxybenzoic acid (pHBA) is considered a mid-range molecule, which currently has an estimated world market of 50,000 t p.a. at a price of around 2,600 US$/t. pHBA is an essential component in liquid crystal polymers which find widespread use in electronics. pHBA is also a precursor for parabens, a class of preservatives in the pharma (Ma et al., 2016) and cosmetics industries (Matwiejczuk et al., 2020). Therefore, the production of para-hydroxybenzoic acid is highly sought after, as a precursor for high performance bioplastics (Polyesters like PET, Polyarylates like Vectran). Because methane is a currently cheap and abundant feedstock, biotechnological production from methane (natural gas/biogas) or from methanol (wood alcohol) may be economically viable as opposed to production from conventional sugar-based carbon-sources.

pHBA has been produced in laboratory scale in biological systems (e.g., E. coli and S. cerevisiae) from sugars, achieving titers (T) of 37 g/L, productivities (R) of over 1.5 g/l/h, and carbon yields (Y) of 66% (Kitade et al., 2018). The titers, productivity, and yield is still too low for commercialization. In E. coli, pHBA is endogenously produced from glucose and is produced as a minor metabolite that is excreted into the culture medium at levels of less than 2 mg/L. However, the amounts of pHBA endogenously produced by E. coli are not optimal for commercial production. Accordingly, there is a need for a better microbial system for the in vivo production of para-hydroxybenzoic acid (pHBA) with titers, yield, and productivity that are commercially relevant.

### SUMMARY

The present disclosure provides a novel genetically engineered microorganism for the commercial production of para-hydroxybenzoic acid (pHBA) from C1 substrate (e.g., methanol and/or methane) using a novel bacterial strain (e.g., Methylomicrobium alcahphilum) that generates enhanced pHBA titer and yield when compared to production in bacterial strains used in the art. pHBA is a precursor and feedstock for various industrially relevant chemicals, including aromatic bioplastics.

In one aspect, the present disclosure provides a method of producing para-hydroxybenzoic acid (pHBA) or a derivative thereof. The method comprises culturing the recombinant microorganism in a fermentation broth; adding a carbon source to the fermentation broth; and isolating the pHBA from the fermentation broth. In some embodiments, the recombinant microorganism comprises a genetically engineered pathway expressing at least one nucleic acid sequence encoding a polypeptide selected from: an exogenous chorismate pyruvate lyase of EC 5.4.4.2 or EC 4.1.3.40; an exogenous 3-deoxy-D-arabino-heptulosonate-7-phosphate (DAHP) synthase of EC 4.1.2.15, or EC 2.5.1.54; an exogenous shikimate kinase of EC 2.7.1.71; or an exogenous 3-dehydroquinate dehydratase (DHQ dehydratase) of EC 4.2.1.10.

In some embodiments, the exogenous DAHP comprises a feedback-inhibition resistant mutation. In some embodiments, the exogenous polypeptide encoded by the nucleic acid is derived from an organism selected from S. cerevisiae, Escherichia coli, Corynebacterium glutamicum, Pseudomonas putida, Providencia rustigianii, Bacillus subtilis, Clostridium autoethanogenum, Clostridium ljungdahlii, Clostridium ragsdalei, Listeria monocytogenes, Streptomyces coelicolor, Propionibacterium freudenreichii, Propionibacterium shermanii, Cronobacter sakazakii, Methylococcus capsulatus, Methylotuvimicrobium buryatense, Methylomicrobium alcaliphilum, Methylobacterium extorquens, Methylotuvimicrobium album, or a combination of any two or more thereof.

In some embodiments, the chorismate pyruvate lyase comprises amino acid sequence of SEQ ID NO: 4 or 5; the DAHP synthase comprises amino acid sequence of SEQ ID NO: 1; the shikimate kinase comprises amino acid sequence of SEQ ID NO: 3; and the DHQ dehydratase comprises amino acid sequence of SEQ ID NO: 2. In some embodiments, the nucleic acid sequence of the genetically engineered pathway comprises a nucleic acid sequence selected from SEQ ID NOs: 6, 45, 52, 53, 54, 55, 56, 57, 58, 59, 60, 61, 62, 63, 64, 65, 66, 67, or a combination of any two or more thereof. In some embodiments, the recombinant microorganism comprises an exogenous amino acid sequence comprising SEQ ID NOs: 1, 2, 3, 4, 5 or a combination of any two or more thereof.

In one aspect, the present disclosure provides a recombinant microorganism for producing a para-hydroxybenzoic acid (pHBA) or a derivative thereof, said recombinant microorganism comprising a genetically engineered pathway expressing at least one nucleic acid sequence encoding a polypeptide selected from: an exogenous chorismate pyruvate lyase of EC 5.4.4.2 or EC 4.1.3.40; an exogenous 3-deoxy-D-arabino-heptulosonate-7-phosphate (DAHP) synthase of EC 4.1.2.15, or EC 2.5.1.54; an exogenous shikimate kinase of EC 2.7.1.71; or an exogenous 3-dehydroquinate dehydratase (DHQ dehydratase) of EC 4.2.1.10.

In some embodiments, the recombinant microorganism is selected from Methylococcus capsulatus, Methylotuvimicrobia, Methylotuvimicrobium buryatense, Methylomicrobium alcaliphilum, Methylotuvimicrobium album, or Methylobacterium extorquens.

In some embodiments, the recombinant microorganism produces pHBA in vivo when grown in a fermentation broth in the presence of a carbon source. In some embodiments, the carbon source comprises methane, methanol, ethanol, carbon monoxide, carbon dioxide, formic acid, or a combination of any two or more thereof.

In some embodiments, the recombinant microorganism comprises a nucleic acid sequence set forth in SEQ ID NO: 6 and a nucleic acid sequence encoding the polypeptide of selected from SEQ ID NO:4; SEQ ID NO: 5; SEQ ID NOs: 1 and 4; SEQ ID NOs: 1 and 5; SEQ ID NOs: 2 and 4; SEQ ID NOs: 2 and 5; SEQ ID NOs: 3 and 4; SEQ ID NOs: 3 and 5; SEQ ID NOs: 1, 3, and 4; SEQ ID NOs: 1, 3, and 5; SEQ ID NOs: 1, 2, and 4 SEQ ID NOs: 1, 2, and 5; SEQ ID NOs: 2, 3, and 4; SEQ ID NOs: 2, 3, and 5; SEQ ID NOs: 1, 2, 3, and 4; or SEQ ID NOs: 1, 2, 3, and 5.

In some embodiments, the recombinant microorganism comprises a nucleic acid sequence set forth in SEQ ID NO: 45.

In one aspect, the present disclosure provides a vector comprising a nucleic acid sequence set forth in SEQ ID NOs: 6, 45, 52, 53, 54, 55, 56, 57, 58, 59, 60, 61, 62, 63, 64, 65, 66, 67, or a combination of any two or more thereof. In some embodiments, the vector is expressed in a microorganism. In some embodiments, the microorganism is used in a method of producing para-hydroxybenzoic acid (pHBA) or a derivative thereof, the method comprising: culturing the recombinant microorganism expressing the vector in a fermentation broth; adding a carbon source to the fermentation broth; and isolating the pHBA from the fermentation broth.

In one or more embodiments, a microbial cell factory is constructed by genetically modifying the bacterium Methylomicrobium alcahphilum 20Z to convert methanol and methane into para-hydroxybenzoic acid, a precursor, and feedstock for various industrially relevant chemicals, including aromatic bioplastics.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIG. 1 shows a schematic illustration of a polyethylene terephthalate biosynthetic pathway from methane by a methanotrophic organisms.

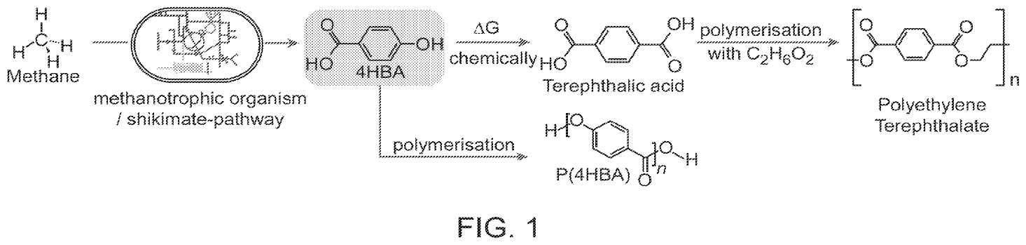

FIGS. 2A-B show a schematic illustration of the genetically engineered shikimate/chorismate pathway of the present disclosure for the conversion of methane to para-hydroxybenzoic acid (4-pHBA) (FIG. 2A); and further show a schematic representation of a single vector driven by a single promoter encoding an embodiment of the genetically engineered pathway of the present disclosure (FIG. 2B). The illustrated pathway is adapted from E. coli shikimate pathway.

FIG. 3 shows a bar graph illustrating the maximum per-biomass yield of para-hydroxybenzoic acid (pHBA) and the production rate of pHBA when the starting material is methanol or methane.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As utilized herein with respect to numerical ranges, the terms “approximately,” “about,” “substantially,” and similar terms will be understood by persons of ordinary skill in the art and will vary to some extent depending upon the context in which it is used. If there are uses of the terms that are not clear to persons of ordinary skill in the art, given the context in which it is used, the terms will be plus or minus 10% of the disclosed values. When “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or illustrative language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the term “about” modifying the quantity of something refers to variation in the numerical quantity that can occur, for example, through typical measuring and liquid handling procedures used for making concentrates or solutions in the real world; through inadvertent error in these procedures; through differences in the manufacture, source, or purity of the ingredients employed to make the compositions or to carry out the methods; and the like. The term “about” also encompasses amounts that differ due to different equilibrium conditions for a composition resulting from a particular initial mixture. Whether or not modified by the term “about,” the claims include equivalents to the quantities. In one embodiment, the term “about” means within 10% of the reported numerical value, alternatively within 5% of the reported numerical value.

As used herein, the term “Codon optimization,” or “codon-optimized” means the mutation of a nucleic acid, such as a gene, for optimized or improved translation of the nucleic acid in a particular strain or species. In some embodiments, “codon optimized” or “codon-optimization” means genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA.

Codon optimization may result in faster translation rates or higher translation accuracy. In a preferred embodiment, the genes of are codon optimized for expression in Methylomicrobium alcahphilum. To codon optimize the heterologous sequences for expression in M. alcahphilum, Methylococcus capsulatus was used as proxy for codon-optimisation of the enzymes of the shikimate pathway. M. capsulatus was selected because an exhaustive review of a codon-usage table showed that the codon-usage appeared similar to that of M. alcahphilum. To maximize translation-rate of the polypeptides (e.g. enzymes of the shikimate pathway for heterologous expression) of the present disclosure.

Initiation of translation was further enhanced using synthetic ribosomal binding sites (RBS; SEQ ID NOs: 7-12). These synthetic ribosomal binding sites were also optimized for effective initiation of expression in M. alcahphilum using a web based interface of De Novo DNA. In some embodiments, the RBS were optimized, and Methylomicrobium album was used as a proxy for RBS and operon design. Salis et al., Methods Enzymol. 498:19-42 (2011).

As used herein, the term “effective titer” means the total amount of a para-hydroxybenzoic acid produced by fermentation or para-hydroxybenzoic acid derivatives produced per liter of fermentation medium. For example, the effective titer of para-hydroxybenzoic acid in a unit volume of a fermentation includes: (i) the amount of para-hydroxybenzoic acid in the fermentation medium; (ii) the amount of para-hydroxybenzoic acid recovered from the organic extractant; (iii) the amount of para-hydroxybenzoic acid recovered from the gas phase, if gas stripping is used; and (iv) the para-hydroxybenzoic acid in either the organic or aqueous phase. As used herein, the term “effective rate” is the effective titer divided by the fermentation time. As used herein, the term “effective yield” is the total grams of product para-hydroxybenzoic acid produced per gram of carbon source (e.g., CO, CO₂, methane or methanol) consumed.

As used herein, the term “mutated” refers to a nucleic acid or protein that has been modified in the microorganism compared to the wild-type or parental microorganism from which the microorganism is derived. In some embodiments, the mutation may be a deletion, insertion, or substitution in a gene encoding an enzyme. In some embodiments, the mutation may be a deletion, insertion, or substitution of one or more amino acids in an enzyme. In particular, a “disruptive mutation” is a mutation that reduces or eliminates (i.e., “disrupts”) the expression or activity of a gene or enzyme. The disruptive mutation may partially inactivate, fully inactivate, or delete the gene or enzyme. The disruptive mutation may be a knockout (KO) mutation. The disruptive mutation may be any mutation that reduces, prevents, or blocks the biosynthesis of a product produced by an enzyme. The disruptive mutation may include, for example, a mutation in a gene encoding an enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, siRNA, CRISPR) or protein which inhibits the expression of an enzyme. The disruptive mutation may be introduced using any method known in the art.

“Functionally equivalent variants” include nucleic acids whose sequence varies as a result of codon optimization for a particular microorganism. A functionally equivalent variant of a nucleic acid will preferably have at least approximately 70%, approximately 80%, approximately 85%, approximately 90%, approximately 95%, approximately 98%, or greater nucleic acid sequence identity (percent homology) with the referenced nucleic acid. A functionally equivalent variant of a protein will preferably have at least approximately 70%, approximately 80%, approximately 85%, approximately 90%, approximately 95%, approximately 98%, or greater amino acid identity (percent homology) with the referenced protein. The functional equivalence of a variant nucleic acid or protein may be evaluated using any method known in the art.

In one aspect, a method of producingpara-hydroxybenzoic acid (pHBA) or a derivative thereof is provided, the method comprising: culturing a recombinant microorganism in a fermentation broth, adding a carbon source to the fermentation broth; and isolating the pHBA from the fermentation broth. The recombinant microorganism of the method comprises a genetically engineered pathway expressing at least one nucleic acid sequence encoding a polypeptide selected from an exogenous chorismate pyruvate lyase of EC 5.4.4.2 or EC 4.1.3.40; an exogenous 3-deoxy-D-arabino-heptulosonate-7-phosphate (DAHP) synthase of EC 4.1.2.15, or EC 2.5.1.54; an exogenous shikimate kinase of EC 2.7.1.71; or an exogenous 3-dehydroquinate dehydratase (DHQ dehydratase) of EC 4.2.1.10.

para-hydroxybenzoic acid (p-PHBA) is a key monomer in the synthesis of liquid crystalline polymers (LCPs) and the manufacture of paraben preservatives and other products. para-hydroxybenzoic acid (PHBA) is produced in two different ways in vivo. The first pathway is the “shikimate pathway” utilized in prokaryotes, which induces the conversion of chorismate to para-hydroxybenzoate through the action of chorismate pyruvate lyase. The second pathway is utilized in mammalian systems and induces induction of para-hydroxybenzoate by derivation of tyrosine or phenylalanine. In bacteria, fungi and plants, pHBA is almost exclusively generated via the shikimate biosynthetic pathway.

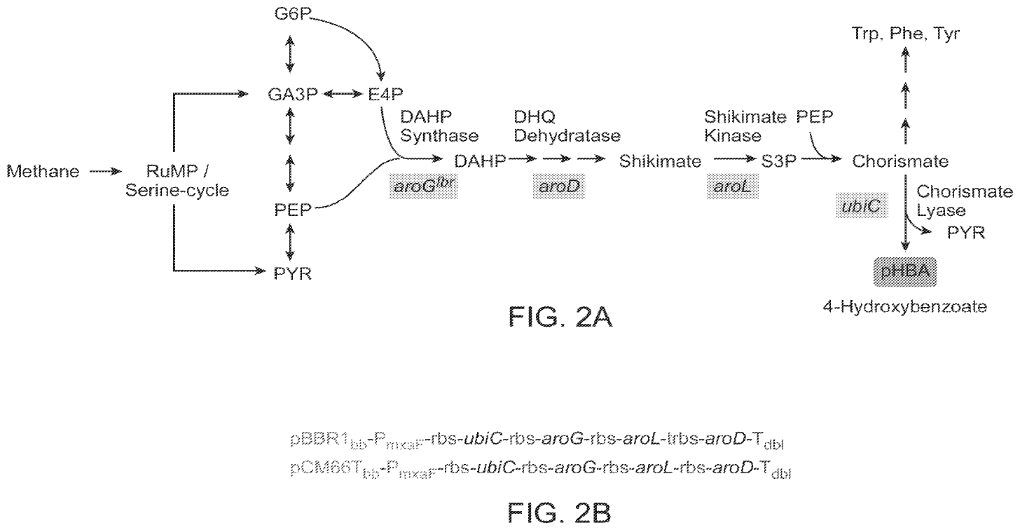

The shikimate pathway is the central metabolic route leading to formation of tryptophan (TRP), tyrosine (TYR), and phenylalanine (PHE). The shikimate pathway starts with the condensation of intermediates of glycolysis and pentosephosphate-pathway, phosphoenolpyruvate (PEP), and erythrose-4-phosphate (E4P), respectively, which enter the pathway through a series of condensation and redox reactions via 3-deoxy-d-arabino-heptulosonate-7-phosphate (DAHP) synthase, 3-dehydroquinate (DHQ), and 3-dehydroshikimate (DHS) to generate shikimate. From there, shikimate is converted to shikimate 3-phosphate through the activity of the shikimate kinase, which is then converted to chorismate. Chorismate is then metabolized to para-hydroxybenzoic acid by the chorismate pyruvate lyase. (FIG. 2A).

In some embodiments, the method for producing a para-hydroxybenzoic acid (pHBA) or a derivative thereof of the present disclosure comprises a recombinant microorganism expressing a genetically engineered pathway expressing at least one nucleic acid sequence encoding an enzyme of the shikimate pathway. In some embodiments, the method of the present disclosure comprises a recombinant microorganism expressing a genetically engineered pathway expressing at least one nucleic acid sequence encoding an exogenous chorismate pyruvate lyase of EC 5.4.4.2 or EC 4.1.3.40 and an exogenous 3-deoxy-D-arabino-heptulosonate-7-phosphate (DAHP) synthase of EC 4.1.2.15, or EC 2.5.1.54; an exogenous shikimate kinase of EC 2.7.1.71; or an exogenous 3-dehydroquinate dehydratase (DHQ) of EC 4.2.1.10.

In some embodiments, the exogenous (DAHP) synthase, shikimate kinase, DHQ dehydratase, or chorismate pyruvate lyase polypeptide is is derived from an organism selected from S. cerevisiae, Escherichia coli, Corynebacterium glutamicum, Pseudomonas putida, Providencia rustigianii, Bacillus subtilis, Clostridium autoethanogenum, Clostridium ljungdahlii, Clostridium ragsdalei, Listeria monocytogenes, Streptomyces coelicolor, Propionibacterium freudenreichii, Propionibacterium shermanii, Cronobacter sakazakii, Methylococcus capsulatus, Methylotuvimicrobium buryatense, Methylomicrobium alcaliphilum, Methylobacterium extorquens, Methylotuvimicrobium album, or a combination of any two or more thereof.

In some embodiments, the nucleic acid encodes a chorismate pyruvate lyase selected from P. rustigianii UbiC or C. sakazakii UbiC. In some embodiments, the nucleic acid encodes an E. coli DAHP synthase (e.g., AroG). In some embodiments, the nucleic acid encoding an E. coli shikimate kinase (e.g., AroL). In some embodiments, the nucleic acid encodes an E. coli DHQ dehydratase (e.g., AroD). In some embodiments, the nucleic acid encodes two or more of DAHP synthase, the DHQ dehydratase, the shikimate kinase, or the chorismate pyruvate lyase. The recombinant microorganism: expresses an exogenous chorismate pyruvate lyase of EC 5.4.4.2 or EC 4.1.3.40; or expresses an exogenous E. coli UbiC; and produces para-hydroxybenzoic acid.

In some embodiments, the method for producing a para-hydroxybenzoic acid (pHBA) or a derivative thereof of the present disclosure comprises a genetically engineered pathway expressing at least one nucleic acid sequence encoding an exogenous an exogenous 3-deoxy-D-arabino-heptulosonate-7-phosphate (DAHP) synthase of EC 4.1.2.15, or EC 2.5.1.54. DAHP synthase catalyses the first committed step in the shikimate pathway, in which erythrose-4-phosphate and phosphoenolpyruvate are converted to 3-deoxy-D-arabinoheptosonate-7-phosphate (FIG. 2A). Amplification of DAHP Synthase activity is an essential strategy to overproduce aromatic compounds and shikimate. For example, Escherichia coli contains three DAHP synthase isozymes (aroF, aroG, aroH), which are each feedback inhibited by one of the three aromatic amino acids (TYR, PHE, TRP). In E. coli, AroG contributes about 80% of the overall DAHP synthase activity. AroF about 15%, and the remaining activity corresponds to AroH DAHP synthase.

In some embodiments, the method for producing a para-hydroxybenzoic acid (pHBA) or a derivative thereof of the present disclosure comprises a nucleic acid encoding an E. coli DAHP synthase. In some embodiments, the recombinant microorganism comprises a nucleic acid encoding an E. coli aroF, aroG, or aroH. In some embodiments, the recombinant microorganism comprises a nucleic acid encoding an E. coli DAHP synthase. The activity of DAHP synthase is subject to feedback inhibition by aromatic amino acids such as tryptophan, phenylalanine, tyrosine as described for E. coli (Hu et al. J. Basic Microbiol., 43:399-406 (2003). To prevent these amino acid from inhibiting the genetically engineered pathway, a mutant DAHP synthase was generated.

In some embodiments, the exogenous DAHP synthase comprises a feedback-inhibition resistant mutation (feedback insensitive mutant; feedback-inhibition DAHP synthase). In some embodiments, the exogenous DAHP synthase mutation comprises: a feedback-inhibition resistant substitution; a substitution at position 180 of the wild-type amino acid sequence of DAHP synthase; a serine to phenylalanine mutation at position 180 of the wild-type amino acid sequence of DAHP synthase; or amino acid sequence set forth in SEQ ID NO: 1.

The feedback-insensitive DAHP synthase reduces the risk of flux to chorismate-derived products being reduced by this feedback inhibition. By way of example, the nucleic acid encoding a DAHP synthase may be derived from Escherichia coli, Clostridium beijerinckii, or Saccharomyces cerevisiae. In one embodiment, the DAHP synthase may be feedback-insensitive DAHP synthase from Escherichia coli, having amino acid sequence of SEQ ID NO: 1. The feedback-insensitive DAHP synthase may be introduced on the same vector as a gene encoding one of the aforementioned enzymes or on a different vector. The feedback-inhibiton insensitive DAHP synthase may have its own promoter or may follow a promoter from methanol dehydrogenase promoter (PmxaF) of M. extorquens, ribulokinase promoter (araBp) of E. coli “P_{BAD}”, β-galactosidase promoter (lacZp) of E. coli “P_{lac}”, or bacteriophage lambda promoter (λP_{L}).

In some embodiments, the method for producing a para-hydroxybenzoic acid (pHBA) or a derivative thereof of the present disclosure comprises a nucleic acid encoding a DAHP synthase gene is derived from any microorganism having such a gene. In some embodiments, the DAHP synthase is derived from an organism selected from S. cerevisiae, Escherichia coli, Corynebacterium glutamicum, Pseudomonas putida, Providencia rustigianii, Bacillus subtilis, Clostridium autoethanogenum, Clostridium ljungdahlii, Clostridium ragsdalei, Listeria monocytogenes, Streptomyces coelicolor, Propionibacterium freudenreichii, Propionibacterium shermanii, Cronobacter sakazakii, Methylococcus capsulatus, Methylotuvimicrobium buryatense, Methylomicrobium alcahphilum, Methylobacterium extorquens, or Methylotuvimicrobium album.

In some embodiments, the DAHP synthase gene is derived from Escherichia coli, Klebsiella oxytoca, Citrobacter freundii, P. rustigianii, C. sakazakii, or any other microorganism having a DAHP synthase gene. In some embodiment, the DAHP synthase gene is AroG and comprises a nucleotide sequence set forth in SEQ ID NO: 53, 57, 61, 65, or a codon-optimized or functionally equivalent variant thereof.

In some embodiments, the method for producing a para-hydroxybenzoic acid (pHBA) or a derivative thereof comprises a genetically engineered pathway expressing at least one nucleic acid sequence encoding an exogenous 3-dehydroquinate dehydratase (DHQ dehydratase) of EC 4.2.1.10. DHQ dehydratase catalyzes the conversion of 3-dehydroquinic acid to 3-dehydroshikimic acid of the shikimate pathway, which is the third step in the shikimate pathway. Overexpression of DHQ dehydratase enhanced the transformation of quinic acid into shikimic acid. In some embodiments, the DHQ dehydratase of the present disclosure is an E. coli AroD. In some embodiments, the DHQ dehydratase comprises amino acid sequence of SEQ ID NO: 2. In some embodiments, the DHQ dehydratase is introduced on the same vector as a gene encoding one of the aforementioned enzymes or on a different vector. In some embodiments, the DHQ dehydratase is driven by its own promoter. In some embodiments, the DHQ dehydratase is driven by a promoter selected from methanol dehydrogenase promoter (PmxaF) of M. extorquens, ribulokinase promoter (araBp) of E. coli “P_{BAD}”, β-galactosidase promoter (lacZp) of E. coli “P_{lac}”, or bacteriophage lambda promoter (λP_{L}). In some embodiments, the DHQ dehydratase is tagged and/or is driven by ribosomal binding site.

In some embodiments, the method for producing a para-hydroxybenzoic acid (pHBA) or a derivative thereof of the present disclosure comprises a nucleic acid encoding a DHQ dehydratase gene derived from any microorganism having such a gene. In some embodiments, the DHQ dehydratase is derived from S. cerevisiae, Escherichia coli, Corynebacterium glutamicum, Pseudomonas putida, Providencia rustigianii, Bacillus subtilis, Clostridium autoethanogenum, Clostridium ljungdahlii, Clostridium ragsdalei, Listeria monocytogenes, Streptomyces coelicolor, Propionibacterium freudenreichii, Propionibacterium shermanii, Cronobacter sakazakii, Methylococcus capsulatus, Methylotuvimicrobium buryatense, Methylomicrobium alcahphilum, Methylobacterium extorquens, or Methylotuvimicrobium album. In some embodiments, the DHQ dehydratase gene is derived from Escherichia coli, Klebsiella oxytoca, Citrobacter freundii, P. rustigianii, C. sakazakii, or any other microorganism having a DHQ dehydratase gene. In some embodiment, the DHQ dehydratase gene is AroD and comprises a nucleotide sequence set forth in SEQ ID NO: 55, 59, 63, 67, or a codon-optimized or functionally equivalent variant thereof.

In some embodiments, the method for producing a para-hydroxybenzoic acid (pHBA) or a derivative thereof comprises a genetically engineered pathway expressing at least one nucleic acid sequence encoding an exogenous shikimate kinase of EC 2.7.1.71. Shikimate kinase catalyzes the ATP-dependent phosphorylation of shikimate to form shikimate 3-phosphate. This reaction is the fifth step of the shikimate pathway, which is used by plants and bacteria to synthesize the common precursor of aromatic amino acids and secondary metabolites. In E. coli, the shikimate kinase I (aroK) and II (aroL) are considered to be the rate-limiting enzyme in the shikimate pathway. Rodriguez et al., Microb Cell Fact. 13(1): 126-(2014). Amplification of the shikimate kinase activity can relieve the rate-limiting steps in the production of shikimate and aromatic compounds. Shikimate is a key intermediate in the biosynthetic aromatic pathway.

In some embodiments, the exogenous shikimate kinase is an E. coli shikimate kinase. In some embodiments, the exogenous shikimate kinase is AroL or AroK. In some embodiments, the shikimate kinase comprises amino acid sequence of SEQ ID NO: 3. In some embodiments, the shikimate kinase is introduced on the same vector as a gene encoding one of the aforementioned enzymes or on a different vector. In some embodiments, the shikimate kinase is driven by its own promoter. In some embodiments, the shikimate kinase is driven by a promoter selected from methanol dehydrogenase promoter (PmxaF) of M. extorquens, ribulokinase promoter (araBp) of E. coli “PBAD”, β-galactosidase promoter (lacZp) of E. coli “Plac”, or bacteriophage lambda promoter (λPL). In some embodiments, the shikimate kinase is tagged and/or is driven by ribosomal binding site.

In some embodiments, the method for producing a para-hydroxybenzoic acid (pHBA) or a derivative thereof comprises a nucleic acid encoding an exogenous shikimate kinase gene derived from any microorganism having such a gene. In some embodiments, the shikimate gene is derived from Escherichia coli, Klebsiella oxytoca, Citrobacter freundii, P. rustigianii, C. sakazakii, or any other microorganism having a shikimate kinase gene. In some embodiments, the shikimate kinase is derived from S. cerevisiae, Escherichia coli, Corynebacterium glutamicum, Pseudomonas putida, Providencia rustigianii, Bacillus subtilis, Clostridium autoethanogenum, Clostridium ljungdahlii, Clostridium ragsdalei, Listeria monocytogenes, Streptomyces coelicolor, Propionibacterium freudenreichii, Propionibacterium shermanii, Cronobacter sakazakii, Methylococcus capsulatus, Methylotuvimicrobium buryatense, Methylomicrobium alcaliphilum, Methylobacterium extorquens, or Methylotuvimicrobium album. In some embodiment, the shikimate kinase gene is AroL and comprises a nucleotide sequence set forth in SEQ ID NO: 54, 58, 62, 66, or a codon-optimized or functionally equivalent variant thereof.

In some embodiments, the method for producing a para-hydroxybenzoic acid (pHBA) or a derivative thereof of the present disclosure comprises a genetically engineered pathway expressing at least one nucleic acid sequence encoding an exogenous chorismate pyruvate lyase of EC 5.4.4.2 or EC 4.1.3.40. The Chorismate pyruvate lyase enzyme catalyzes the conversion of chorismate to para-hydroxybenzoic acid and pyruvate in the first committed step of ubiquinone biosynthesis. The elimination of pyruvate from chorismate results in the formation of pHBA. This aromatizing reaction is the first committed step in ubiquinone biosynthesis in E. coli and Salmonella enterica and is catalyzed by the chorismate pyruvate lyase. In E. coli, chorismate pyruvate lyase is encoded the ubiC gene.

In some embodiments, the chorismate pyruvate lyase is derived from any microorganism having such an enzyme. In some embodiments, the chorismate pyruvate lyase is a UbiC enzyme. In some embodiments, the Ubic is derived from Escherichia coli, Klebsiella oxytoca, P. rustigianii, Citrobacter freundii, C. sakazakii or any other microorganism having a UbiC enzyme. In some embodiments, the chorismate pyruvate lyase is derived from S. cerevisiae, Escherichia coli, Corynebacterium glutamicum, Pseudomonas putida, Providencia rustigianii, Bacillus subtilis, Clostridium autoethanogenum, Clostridium ljungdahlii, Clostridium ragsdalei, Listeria monocytogenes, Streptomyces coelicolor, Propionibacterium freudenreichii, Propionibacterium shermanii, Cronobacter sakazakii, Methylococcus capsulatus, Methylotuvimicrobium buryatense, Methylomicrobium alcaliphilum, Methylobacterium extorquens, or Methylotuvimicrobium album.

In some embodiments, the UbiC enzyme is derived from C. sakazakii and comprises an amino acid sequence set forth in SEQ ID NO: 5 or a functionally equivalent variant thereof. In some embodiments, the UbiC enzyme is derived from P. rustigianii and comprises an amino acid sequence set forth in SEQ ID NO: 4 or a functionally equivalent variant thereof. In some embodiments, the exogenous chorismate pyruvate lyase is a P. rustigianii UbiC or C. sakazakii UbiC. In some embodiments, the chorismate pyruvate lyase comprises amino acid sequence of SEQ ID NO: 4 or 5.

In some embodiments, the chorismate pyruvate lyase is introduced on the same vector as a gene encoding one of the aforementioned enzymes or on a different vector. In some embodiments, the chorismate pyruvate lyase is driven by its own promoter. In some embodiments, the chorismate pyruvate lyase is driven by a promoter selected from methanol dehydrogenase promoter (PmxaF) of M. extorquens, ribulokinase promoter (araBp) of E. coli “PBAD”, β-galactosidase promoter (lacZp) of E. coli “Plac”, or bacteriophage lambda promoter (λPL). In some embodiments, the chorismate pyruvate lyase is tagged and/or is driven by ribosomal binding site

In some embodiments, the method for producing a para-hydroxybenzoic acid (pHBA) or a derivative thereof comprises a nucleic acid encoding an exogenous chorismate pyruvate lyase gene derived from any microorganism having such a gene. In some embodiments, the chorismate pyruvate lyase gene is a ubiC gene derived from Escherichia coli, Klebsiella oxytoca, Citrobacter freundii, P. rustigianii, C. sakazakii, or any other microorganism having a ubiC gene. In some embodiments, the chorismate pyruvate lyase gene is ubiC comprises a nucleotide sequence set forth in SEQ ID NO: 52, 56, 60, 64, or a codon-optimized or functionally equivalent variant thereof.

The UbiC enzyme or ubiC gene of the present method may also be modified (e.g., mutated) to enhance solubility, stability, or other gene/enzyme properties. Such modifications may result in increased product titers. One particular modification involves engineering the ubiC gene to express a UbiC enzyme with two surface-active serines instead of cysteines. The serine residues result in less protein aggregation and, in turn, improved solubility. Accordingly, in a particular embodiment, the UbiC enzyme comprises a mutation to replace at least one surface-active cysteine with a serine.

Production of para-hydroxybenzoic acid (PHB) is increased by overexpression of at least of an exogenous an exogenous chorismate pyruvate lyase of EC 5.4.4.2 or EC 4.1.3.40; an exogenous feedback-inhibition 3-deoxy-D-arabino-heptulosonate-7-phosphate (DAHP) synthase of EC 4.1.2.15, or EC 2.5.1.54; an exogenous shikimate kinase of EC 2.7.1.71; or an exogenous 3-dehydroquinate dehydratase (DHQ dehydratase) of EC 4.2.1.10. All these genes are codon-optimized variants of Methylococcus capsulatus codon sequences. In a preferred embodiment, of the present disclosure, the exogenous DAHP synthase comprises a S180F substitution that alleviates the feedback inhibition of DAHP synthase by one of three aromatic amino acids (e.g. tyrosine (Tyr), phenylalanine (Phe), or tryptophan (Trp).

In some embodiments, the method for producing a para-hydroxybenzoic acid (pHBA) or a derivative thereof comprises a chorismate pyruvate lyase comprising amino acid sequence of SEQ ID NO: 4 or 5; a DAHP synthase comprising amino acid sequence of SEQ ID NO: 1; a shikimate kinase comprising amino acid sequence of SEQ ID NO: 3; a DHQ dehydratase comprises amino acid sequence of SEQ ID NO: 2, or a combination thereof. In some embodiments, the method for producing a para-hydroxybenzoic acid (pHBA) or a derivative thereof comprises an exogenous amino acid sequence comprising SEQ ID NOs: 1, 2, 3, 4, 5 or a combination of any two or more thereof.

In some embodiments, the method for producing a para-hydroxybenzoic acid (pHBA) or a derivative thereof comprises a genetically engineered pathway encoded by a single vector driven by a single promoter. In some embodiments, the single vector comprises p_{mxaF}-ubiC; p_{mxaF}-UbiC-aroG; p_{mxaF}-ubiC-aroL; p_{mxaF}-ubiC-aroD; p_{mxaF}-ubiC-aroG-aroL; p_{mxaF}-ubiC-aroG-aroD; p_{mxaF}-ubiC-aroL-aroD; or p_{mxaF}-UbiC-aroG-aroL-aroD.

In some embodiments, the method for producing a para-hydroxybenzoic acid (pHBA) or a derivative thereof comprises a single vector driven by a single promoter. In some embodiments, the promoter is a constitutive promoter or an inducible promoter. In some embodiments, the promotor is selected from a M. extorquens methanol dehydrogenase promoter (PmxaF), an E. coli (PBAD) ribulokinase promoter (araBp), an E. coli (Plac) β-galactosidase promoter (lacZp), or a bacteriophage lambda promoter (λPL); or a promotor encoded by a nucleic acid sequence set forth in SEQ ID NO: 6. In some embodiments, the vector comprises at least two, at least three, at least four, or at least five nucleic sequences each encoding a polypeptide.

In some embodiments, each nucleic acid encoding a polypeptide of the shikimate pathway as described herein is conjugated to a nucleic acid sequence encoding a ribosomal binding site and/or a tag protein. In some embodiments, the nucleic acid sequence of the ribosomal binding site is selected from SEQ ID NO: 7, 8, 9, 10, 11, or 12. In some embodiments, the tag is encoded by a nucleic acid sequence selected from SEQ ID NO: 21, 22, 23, or 24. In some embodiments, each nucleic acid encoding a polypeptide is conjugated to a nucleic acid encoding ribosomal binding site (RBS) and a tag protein. In some embodiments, the single vector further comprises a spacer sequence between each nucleic acid encoding a polypeptide as described herein. In some embodiments, the spacer is encoded by a nucleic acid sequence selected from SEQ ID NO: 13, 14, 15, 16, 17, 18, or 19.

In some embodiments, the method for producing a para-hydroxybenzoic acid (pHBA) or a derivative thereof comprises a genetically engineered pathway expressing at least one nucleic acid sequence encoding a polypeptide comprising a nucleic acid sequence selected from SEQ ID NOs: 6, 45, 52, 53, 54, 55, 56, 57, 58, 59, 60, 61, 62, 63, 64, 65, 66, 67, or a combination of any two or more thereof. In some embodiments, the method for producing a para-hydroxybenzoic acid (pHBA) or a derivative thereof comprises a nucleic acid sequence set forth in SEQ ID NO: 45.

In some embodiments, the method for producing a para-hydroxybenzoic acid (pHBA) or a derivative thereof comprises a nucleic acid sequence set forth in SEQ ID NO: 6 and a nucleic acid sequence encoding the polypeptide selected from SEQ ID NO:4; SEQ ID NO: 5; SEQ ID NOs: 1 and 4; SEQ ID NOs: 1 and 5; SEQ ID NOs: 2 and 4; SEQ ID NOs: 2 and 5; SEQ ID NOs: 3 and 4; SEQ ID NOs: 3 and 5; SEQ ID NOs: 1, 3, and 4; SEQ ID NOs: 1, 3, and 5; SEQ ID NOs: 1, 2, and 4; SEQ ID NOs: 1, 2, and 5; SEQ ID NOs: 2, 3, and 4; SEQ ID NOs: 2, 3, and 5; SEQ ID NOs: 1, 2, 3, and 4; or SEQ ID NOs: 1, 2, 3, and 5.

In some embodiments, the method for producing a para-hydroxybenzoic acid (pHBA) or a derivative thereof comprises a recombinant microorganism selected from Methylococcus capsulatus, Methylotuvimicrobia, Methylotuvimicrobium buryatense, Methylomicrobium alcaliphilum, Methylotuvimicrobium album, or Methylobacterium extorquens. In some embodiments, the recombinant microorganism is Methylomicrobium alcaliphilum 20Z. In some embodiments, at least one nucleic acid sequence encoding a polypeptide as described herein is codon optimized for expression in Methylomicrobium alcaliphilum 20Z.

In some embodiments, the method for producing a para-hydroxybenzoic acid (pHBA) or a derivative thereof comprises a recombinant C₁-metabolizing microorganism. In some embodiments, the recombinant microorganism is a recombinant methanogenic organism (e.g., methanotroph).

In some embodiments, the method for producing pHBA or a derivative thereof comprises adding a carbon source to the fermentation broth comprising a recombinant microorganism as described herein. In some embodiments, the method for producing a para-hydroxybenzoic acid (pHBA) or a derivative thereof comprises culturing the recombinant microorganism as described herein in a fermentation broth (“fermented mixture” or “fermentation medium) in the presence of a carbon source. In some embodiments, the recombinant microorganism produces pHBA in vivo when grown in a fermentation broth in the presence of a carbon source.

In some embodiments, the carbon source is a C1-substrate. Illustrative C1 substrates include syngas, methane (CH₄), methanol (CH₃OH), formaldehyde, formic acid (CH₂O₂) or a salt thereof, carbon monoxide (CO), carbon dioxide (CO₂), methylated amines (e.g., methylamine, dimethylamine, trimethylamine, etc.), methylated thiols, methyl halogens (e.g., bromomethane, chloromethane, iodomethane, dichloromethane, etc.), cyanide, or any combination thereof. In some embodiments, the carbon source is carbon dioxide (CO₂), methane (CH₄), methanol (CH₃OH), syngas (i.e. obtained by gasification of coal or refinery residues, gasification of biomass, or reforming of natural gas”), ethanol, carbon monoxide (CO), or formic acid (CH₂O₂), or a combination of any two or more thereof. In some embodiments, the carbon source is methanol, methane, or a combination thereof.

In some embodiments, the substrate generally comprises at least some amount of CO, such as about 1, 2, 5, 10, 20, 30, 40, 50, 60, 70, 80, 90, or 100 mol % CO. The substrate may comprise a range of CO, such as about 20-80, 30-70, or 40-60 mol % CO. Preferably, the substrate comprises about 40-70 mol % CO (e.g., steel mill or blast furnace gas), about 20-30 mol % CO (e.g., basic oxygen furnace gas), or about 15-45 mol % CO (e.g., syngas). In some embodiments, the substrate may comprise a relatively low amount of CO, such as about 1-10 or 1-20 mol % CO. The microorganism typically converts at least a portion of the CO in the substrate to a product.

The substrate may comprise some amount of CO₂. For example, the substrate may comprise about 1-80 or 1-30 mol % CO₂. In some embodiments, the substrate may comprise less than about 20, 15, 10, or 5 mol % CO₂. In another embodiment, the substrate comprises substantially no CO₂. Although the substrate is typically gaseous, the substrate may also be provided in alternative forms. For example, the substrate may be dissolved in a liquid saturated with a CO-containing gas using a microbubble dispersion generator. By way of further example, the substrate may be adsorbed onto a solid support.

The C₁-substrate (carbon source) may be a waste gas obtained as a byproduct of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining processes, coal gasification, electric power production, carbon black production, ammonia production, methanol production, and coke manufacturing. In these embodiments, the substrate and/or C₁-carbon source may be captured from the industrial process before it is emitted into the atmosphere, using any convenient method.

In some embodiments, the syngas metabolizing bacteria is selected from the group consisting of Clostridium autoethanogenum, Clostridium ljungdahli, Clostridium ragsdalei, Clostridium carboxydivorans, Butyridbacterium methylotrophicum, Clostridium woodii, and Clostridium neopropanologen, Corynebacterium glutamicum, Pseudomonas putida, Providencia rustigianii, Bacillus subtilis, Listeria monocytogenes, Streptomyces coelicolor, Propionibacterium freudenreichii, Propionibacterium shermanii, Cronobacter sakazakii, Methylococcus capsulatus, Methylotuvimicrobium buryatense, Methylomicrobium alcaliphilum, Methylobacterium extorquens, or Methylotuvimicrobium album.

In some embodiments, the produced pHBA or derivatives thereof is contained in a fermentation product broth comprising the para-hydroxybenzoic acid (pHBA) produced by a recombinant microorganism as described herein. In some embodiments, the fermentation product broth may have been processed to remove any components such as microorganisms.

In some embodiments, the method for producing a para-hydroxybenzoic acid (pHBA) or a derivative thereof comprises culturing a recombinant microorganism as described herein in a fermentation broth.

In some embodiments, the culture is performed in a bioreactor. In some embodiments, the bioreactor comprises a first growth reactor and a second culture/fermentation reactor. The carbon source (i.e. substrate) may be provided to one or both of these reactors.

In some embodiments, the culture/fermentation is carried out under appropriate conditions for production of the target product (para-hydroxybenzoic acid (pHBA) or a derivative thereof). Suitable reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the carbon source may be controlled to ensure that the concentration of gas in the liquid phase does not become limiting, since products may be consumed by the culture under gas-limited conditions.

Operating a bioreactor at elevated pressures may allow for an increased rate of gas mass transfer from the gas phase to the liquid phase. Accordingly, the culture/fermentation may be performed at pressures higher than atmospheric pressure.

In some embodiments, the method for producing a para-hydroxybenzoic acid (pHBA) or a derivative thereof comprises isolating the pHBA from the fermentation broth. The pHBA may be separated or purified from a fermentation broth using any method or combination of methods known in the art, including, for example, fractional distillation, evaporation, pervaporation, gas stripping, phase separation, ion exchange chromatograph, and extractive fermentation, including for example, liquid-liquid extraction. In certain embodiments, the para-hydroxybenzoic acid (pHBA) and/or derivatives thereof are recovered from the fermentation broth by continuously removing a portion of the broth from the bioreactor, separating microbial cells from the broth (conveniently by filtration), and recovering one or more of the para-hydroxybenzoic acid (pHBA) or derivatives thereof from the broth.

In one aspect, the present disclosure provides a method of producing para-hydroxybenzoic acid (pHBA) or a derivative thereof, the method comprising: culturing a recombinant microorganism expressing a vector comprising a nucleic acid sequence set forth in SEQ ID NOs: 6, 45, 52, 53, 54, 55, 56, 57, 58, 59, 60, 61, 62, 63, 64, 65, 66, 67, or a combination of any two or more thereof in a fermentation broth; adding a carbon source to the fermentation broth; and isolating the pHBA from the fermentation broth. In some embodiments, the vector comprises a nucleic acid sequence set forth in SEQ ID NO: 45.

In one aspect, a recombinant microorganism for producing apara-hydroxybenzoic acid (pHBA) or a derivative thereof is provided, where the recombinant microorganism includes a genetically engineered pathway expressing at least one nucleic acid sequence encoding a polypeptide selected from: an exogenous chorismate pyruvate lyase of EC 5.4.4.2 or EC 4.1.3.40; an exogenous 3-deoxy-D-arabino-heptulosonate-7-phosphate (DAHP) synthase of EC 4.1.2.15, or EC 2.5.1.54; an exogenous shikimate kinase of EC 2.7.1.71; or an exogenous 3-dehydroquinate dehydratase (DHQ dehydratase) of EC 4.2.1.10.

Heterotrophic microorganisms such as E. coli and S. cerevisiae produce relatively high levels of ATP through glycolysis. In contrast, microorganisms that use C1-carbon sources (e.g., CO, CO2, methane or methanol) have poor ATP availability. For example, in E. coli, pHBA can be made from glucose and is produced as a minor metabolite and is excreted into the medium at levels of less than 2 mg/L. However, the amounts of pHBA endogenously produced by E. coli are not optimal for commercial production. The biosynthetic pathway in E. coli is shown in FIG. 2A, which is genetically engineered in a heterologous microorganism described in the present disclosure.

In contrast, analysis of the reaction kinetics in a typical C₁-metabolizing microorganism, such as C. autoethanogenum gave a predicted ATP yield when producing pHBA of −0.4 ATP per mol of CO fixed. As such, it would not be expected that any pHBA would be produced due to the energy constraints. Similarly it would not be expected that other chorismate-derived products would be produced by a wild-type/natural C1-metabolizing microorganism due to the metabolic burden of producing such compounds under autotrophic conditions. In some embodiments, the microorganism for producing a pHBA or a derivative thereof is a recombinant C1-metabolizing microorganism. In some embodiments, the recombinant C₁-metabolizing microorganism is cultured with a C₁-substrate feedstock as described herein.

C1 metabolizing microorganisms include bacteria (such as methanotrophs and methylotrophs) and yeast. In certain embodiments, a C1 metabolizing microorganism does not include a photosynthetic microorganism, such as algae. In some embodiments, the C1 metabolizing microorganism is an “obligate C1 metabolizing microorganism,” meaning its sole source of energy are C1 substrates. In further embodiments, a C1 metabolizing microorganism (e.g., methanotroph) is cultured in the presence of a C1 substrate feedstock (i.e., using the C1 substrate as a source of energy). In some embodiments, the C1 metabolizing microorganism is a methanotroph.

One aspect of the present disclosure provide a novel genetically engineered pathway in a recombinant methanotroph microorganism that produces a number of chorismate-derived products, including pHBA from a C₁-substrate (e.g. methane or methanol). The present disclosure provides a novel, practical, economic, and environmental beneficial way of producing pHBA and/or derivatives thereof from industrial waste gases.

As used herein, the term “methanotroph” or “methanohile,” “methanotropic” or “methanotrophic bacteria” means a bacteria or microorganism that is capable of utilizing C1 substrates, such as methane or unconventional natural gas, as its primary or sole carbon and energy source. In some embodiments, a methanotroph is an organism that metabolizes methane as its source of carbon. In some embodiments, a methanotroph is Methylomonas, Methylobacter, Methylococcus, Methylosinus, Methylocystis, Methylomicrobium, Methanomonas, Methylocella, or Methylocapsa.

In some embodiments, the methanotroph is selected from the group consisting of Methylococcus capsulatus Bath strain, Methylomonas methanica 16a (ATCC PTA 2402), Methylosinus trichosporium OB3b (NRRL B-11,196), Methylosinus sporium (NRRL B-11,197), Methylocystis parvus (NRRL B-11,198), Methylomonas methanica (NRRL B-11,199), Methylomonas albus (NRRL B-11,200), Methylobacter capsulatus (NRRL B-11,201), Methylobacterium organophilum (ATCC 27,886), Methylomonas sp AJ-3670 (FERM P-2400), Methylocella silvestris, Methylocella palustris (ATCC 700799), Methylocella tundrae, Methylocystis daltona strain SB2, Methylocystis bryophila, Methylocapsa aurea KYG, Methylacidiphilum infernorum, Methylibium petroleiphilum, and Methylomicrobium alcahphilum. In some embodiments, the methanotrophs include Methylocella, Methylocystis, and Methylocapsa (e.g., Methylocella silvestris, Methylocella palustris, Methylocella tundrae, Methylocystis daltona SB2, Methylocystis bryophila, and Methylocapsa aurea KYG), and Methylobacterium organophilum (ATCC 27,886). In some embodiments, the recombinant microorganism of the present disclosure is selected from Methylococcus capsulatus, Methylotuvimicrobia, Methylotuvimicrobium buryatense, Methylomicrobium alcaliphilum, Methylotuvimicrobium album, or Methylobacterium extorquens. In some embodiments, the recombinant microorganism is Methylomicrobium alcaliphilum 20Z.

In some embodiments, a recombinant microorganism for producing a para-hydroxybenzoic acid (pHBA) or a derivative thereof, comprises a genetically engineered pathway expressing at least one nucleic acid sequence encoding a polypeptide selected from: an exogenous chorismate pyruvate lyase of EC 5.4.4.2 or EC 4.1.3.40; an exogenous 3-deoxy-D-arabino-heptulosonate-7-phosphate (DAHP) synthase of EC 4.1.2.15, or EC 2.5.1.54; an exogenous shikimate kinase of EC 2.7.1.71; or an exogenous 3-dehydroquinate dehydratase (DHQ dehydratase) of EC 4.2.1.10.

DAHP synthase catalyses the first committed step in the shikimate pathway, in which erythrose-4-phosphate and phosphoenolpyruvate are converted to 3-deoxy-D-arabinoheptosonate-7-phosphate (FIG. 2A). Amplification of DAHP Synthase activity is an essential strategy to overproduce aromatic compounds and shikimate. For example, Escherichia coli contains three DAHP synthase isozymes (aroF, aroG, aroH), which are each feedback inhibited by one of the three aromatic amino acids (TYR, PHE, TRP). HQ dehydratase catalyzes the conversion of 3-dehydroquinic acid to 3-dehydroshikimic acid of the shikimate pathway, which is the third step in the shikimate pathway. Overexpression of DHQ dehydratase enhanced the transformation of quinic acid into shikimic acid.

Shikimate kinase catalyzes the ATP-dependent phosphorylation of shikimate to form shikimate 3-phosphate. This reaction is the fifth step of the shikimate pathway, which is used by plants and bacteria to synthesize the common precursor of aromatic amino acids and secondary metabolites. In E. coli, the shikimate kinase I (aroK) and II (aroL) are considered to be the rate-limiting enzyme in the shikimate pathway. Rodriguez et al., Microb Cell Fact. 13(1): 126-(2014). Amplification of the the shikimate kinase activity can relieve the rate-limiting steps in the production of shikimate and aromatic compounds. Shikimate is a key intermediate in the biosynthetic aromatic pathway. Chorismate pyruvate lyase enzyme (EC 4.1.3.40) that catalyzes the conversion of chorismate to para-hydroxybenzoic acid and pyruvate in the first committed step of ubiquinone biosynthesis. The elimination of pyruvate from chorismate results in the formation of p-HBA. This aromatizing reaction is the first committed step in ubiquinone biosynthesis in E. coli and Salmonella enterica and is catalyzed by the chorismate pyruvate lyase. In E. coli, chorismate pyruvate lyase is encoded ubiC gene.

In some embodiments, a recombinant microorganism for producing a para-hydroxybenzoic acid (pHBA) or a derivative thereof of the present disclosure comprises a genetically engineered pathway expressing at least one nucleic acid sequence encoding an exogenous an exogenous 3-deoxy-D-arabino-heptulosonate-7-phosphate (DAHP) synthase of EC 4.1.2.15, or EC 2.5.1.54. In some embodiments, the recombinant microorganism comprises a nucleic acid encoding an E. coli DAHP synthase. In some embodiments, the recombinant microorganism comprises a nucleic acid encoding an E. coli aroF, aroG, or aroH.

The activity of DAHP is subject to feedback inhibition by aromatic amino acids such as tryptophan, phenylalanine, tyrosine as described for E. coli (Hu et al. J. Basic Microbiol., 43:399-406 (2003). The feedback-insensitive DAHP synthase reduces the risk of flux to chorismate-derived products being reduced by this feedback inhibition. In some embodiments, the exogenous DAHP synthase comprises a feedback-inhibition resistant mutation (feedback insensitive mutant; feedback-inhibition DAHP synthase). In some embodiments, the exogenous DAHP synthase comprises: a feedback-inhibition resistant substitution; a substitution at position 180 of the wild-type amino acid sequence of DAHP synthase; a serine to phenylalanine mutation at position 180 of the wild-type amino acid sequence of DAHP synthase; or amino acid sequence set forth in SEQ ID NO: 1.

In one embodiment, the DAHP synthase may be feedback-insensitive DAHP synthase from Escherichia coli, having amino acid sequence of SEQ ID NO: 1. The feedback-insensitive DAHP synthase may be introduced on the same vector as a gene encoding one of the aforementioned enzymes or on a different vector. The feedback-inhibition insensitive DAHP synthase may have its own promoter or may follow a promoter from methanol dehydrogenase promoter (PmxaF) of M. extorquens, ribulokinase promoter (araBp) of E. coli “P_{BAD}”, β-galactosidase promoter (lacZp) of E. coli “P_{lac}”, or bacteriophage lambda promoter (λP_{L}).

In some embodiments, the microorganism includes a nucleic acid encoding an exogenous DAHP synthase gene derived from any microorganism having such a gene. In some embodiments, the DAHP synthase is derived from an organism selected from S. cerevisiae, Escherichia coli, Corynebacterium glutamicum, Pseudomonas putida, Providencia rustigianii, Bacillus subtilis, Clostridium autoethanogenum, Clostridium ljungdahlii, Clostridium ragsdalei, Listeria monocytogenes, Streptomyces coelicolor, Propionibacterium freudenreichii, Propionibacterium shermanii, Cronobacter sakazakii, Methylococcus capsulatus, Methylotuvimicrobium buryatense, Methylomicrobium alcahphilum, Methylobacterium extorquens, or Methylotuvimicrobium album. In some embodiments, the DAHP synthase gene is derived from Escherichia coli, Klebsiella oxytoca, Citrobacter freundii, P. rustigianii, C. sakazakii, or any other microorganism having a DAHP synthase gene. In some embodiment, the DAHP synthase gene is AroG and comprises a nucleotide sequence set forth in SEQ ID NO: 53, 57, 61, 65, or a codon-optimized or functionally equivalent variant thereof.

In some embodiments, a recombinant microorganism for producing a para-hydroxybenzoic acid (pHBA) or a derivative thereof includes a genetically engineered pathway expressing at least one nucleic acid sequence encoding an exogenous 3-dehydroquinate dehydratase (DHQ dehydratase) of EC 4.2.1.10. In some embodiments, the DHQ of the present disclosure is an E. coli AroD. In some embodiments, the DHQ dehydratase comprises amino acid sequence of SEQ ID NO: 2. In some embodiments, the DHQ dehydratase is introduced on the same vector as a gene encoding one of the aforementioned enzymes or on a different vector. In some embodiments, the DHQ dehydratase is driven by its own promoter. In some embodiments, the DHQ dehydratase is driven by a promoter selected from methanol dehydrogenase promoter (PmxaF) of M. extorquens, ribulokinase promoter (araBp) of E. coli “P_{BAD}”, β-galactosidase promoter (lacZp) of E. coli “P_{lac}”, or bacteriophage lambda promoter (λP_{L}). In some embodiments, the DHQ dehydratase is tagged and/or is driven by ribosomal binding site.

In some embodiments, a microorganism includes a nucleic acid encoding an exogenous 3-dehydroquinate dehydratase (DHQ dehydratase) gene derived from any microorganism having such a gene. In some embodiments, the DHQ dehydratase is derived from S. cerevisiae, Escherichia coli, Corynebacterium glutamicum, Pseudomonas putida, Providencia rustigianii, Bacillus subtilis, Clostridium autoethanogenum, Clostridium ljungdahlii, Clostridium ragsdalei, Listeria monocytogenes, Streptomyces coelicolor, Propionibacterium freudenreichii, Propionibacterium shermanii, Cronobacter sakazakii, Methylococcus capsulatus, Methylotuvimicrobium buryatense, Methylomicrobium alcahphilum, Methylobacterium extorquens, or Methylotuvimicrobium album. In some embodiments, the DHQ gene is derived from Escherichia coli, Klebsiella oxytoca, Citrobacter freundii, P. rustigianii, C. sakazakii, or any other microorganism having a DHQ dehydratase gene. In some embodiment, the DHQ dehydratase gene is AroD and comprises a nucleotide sequence set forth in SEQ ID NO: 55, 59, 63, 67, or a codon-optimized or functionally equivalent variant thereof.

In some embodiments, a recombinant microorganism for producing a para-hydroxybenzoic acid (pHBA) or a derivative thereof comprises a genetically engineered pathway expressing at least one nucleic acid sequence encoding an exogenous shikimate kinase of EC 2.7.1.71. In some embodiments, the exogenous shikimate kinase is an E. coli shikimate kinase. In some embodiments, the exogenous shikimate kinase is AroL or AroK. In some embodiments, the shikimate kinase comprises amino acid sequence of SEQ ID NO: 3. In some embodiments, the shikimate kinase is introduced on the same vector as a gene encoding one of the aforementioned enzymes or on a different vector. In some embodiments, the shikimate kinase is driven by its own promoter. In some embodiments, the shikimate kinase is driven by a promoter selected from methanol dehydrogenase promoter (PmxaF) of M. extorquens, ribulokinase promoter (araBp) of E. coli “PBAD”, β-galactosidase promoter (lacZp) of E. coli “Plac”, or bacteriophage lambda promoter (λPL). In some embodiments, the shikimate kinase is tagged and/or is driven by ribosomal binding site.

In some embodiments, the microorganism includes a nucleic acid encoding an exogenous shikimate kinase gene derived from any microorganism having such a gene. In some embodiments, the chorismate pyruvate lyase gene is derived from Escherichia coli, Klebsiella oxytoca, Citrobacter freundii, P. rustigianii, C. sakazakii, or any other microorganism having a shikimate kinase gene. In some embodiments, the shikimate kinase is derived from S. cerevisiae, Escherichia coli, Corynebacterium glutamicum, Pseudomonas putida, Providencia rustigianii, Bacillus subtilis, Clostridium autoethanogenum, Clostridium ljungdahlii, Clostridium ragsdalei, Listeria monocytogenes, Streptomyces coelicolor, Propionibacterium freudenreichii, Propionibacterium shermanii, Cronobacter sakazakii, Methylococcus capsulatus, Methylotuvimicrobium buryatense, Methylomicrobium alcahphilum, Methylobacterium extorquens, or Methylotuvimicrobium album. In some embodiment, the shikimate kinase gene is AroL and comprises a nucleotide sequence set forth in SEQ ID NO: 54, 58, 62, 66, or a codon-optimized or functionally equivalent variant thereof.

In some embodiments, a recombinant microorganism for producing a para-hydroxybenzoic acid (pHBA) or a derivative thereof of the present disclosure comprises a genetically engineered pathway expressing at least one nucleic acid sequence encoding an exogenous chorismate pyruvate lyase of EC 5.4.4.2 or EC 4.1.3.40. In some embodiments, the chorismate pyruvate lyase is derived from any microorganism having a chorismate pyruvate lyase gene. In some embodiments, the chorismate pyruvate lyase is a UbiC enzyme. In some embodiments, the Ubic is derived from Escherichia coli, Klebsiella oxytoca, P. rustigianii, Citrobacter freundii, C. sakazakii or any other microorganism having a UbiC enzyme.

In some embodiments, the chorismate pyruvate lyase is derived from S. cerevisiae, Escherichia coli, Corynebacterium glutamicum, Pseudomonas putida, Providencia rustigianii, Bacillus subtilis, Clostridium autoethanogenum, Clostridium ljungdahlii, Clostridium ragsdalei, Listeria monocytogenes, Streptomyces coelicolor, Propionibacterium freudenreichii, Propionibacterium shermanii, Cronobacter sakazakii, Methylococcus capsulatus, Methylotuvimicrobium buryatense, Methylomicrobium alcaliphilum, Methylobacterium extorquens, or Methylotuvimicrobium album.

In one embodiment, the UbiC enzyme is derived from C. sakazakii and comprises an amino acid sequence set forth in SEQ ID NO: 5 or a functionally equivalent variant thereof. In one embodiment, the UbiC enzyme is derived from P. rustigianii and comprises an amino acid sequence set forth in SEQ ID NO: 4 or a functionally equivalent variant thereof. In some embodiments, the exogenous chorismate pyruvate lyase is a P. rustigianii UbiC or C. sakazakii UbiC. In some embodiments, the chorismate pyruvate lyase comprises amino acid sequence of SEQ ID NO: 4 or 5. In some embodiment, the horismate pyruvate lyase gene is ubiC comprises a nucleotide sequence set forth in SEQ ID NO: 52, 56, 60, 64, or a codon-optimized or functionally equivalent variant thereof.

In some embodiments, the chorismate pyruvate lyase is introduced on the same vector as a gene encoding one of the aforementioned enzymes or on a different vector. In some embodiments, the chorismate pyruvate lyase is driven by its own promoter. In some embodiments, the chorismate pyruvate lyase is driven by a promoter selected from methanol dehydrogenase promoter (PmxaF) of M. extorquens, ribulokinase promoter (araBp) of E. coli “PBAD”, β-galactosidase promoter (lacZp) of E. coli “Plac”, or bacteriophage lambda promoter (λPL). In some embodiments, the chorismate pyruvate lyase is tagged and/or is driven by ribosomal binding site.

The UbiC enzyme or ubiC gene may also be modified (e.g., mutated) to enhance solubility, stability, or other gene/enzyme properties. Such modifications may result in increased product titers. One particular modification involves engineering the ubiC gene to express a UbiC enzyme with two surface-active serines instead of cysteines. The serine residues result in less protein aggregation and, in turn, improved solubility. Accordingly, in a particular embodiment, the UbiC enzyme comprises a mutation to replace at least one surface-active cysteine with a serine. In some embodiments, introduction of an exogenous chorismate pyruvate lyase (e.g., ubiC) or a nucleic acid encoding an exogenous chorismate pyruvate lyase (e.g., ubiC) in a recombinant of the microorganism described herein results in the production of para-hydroxybenzoic acid, a chorismate-derived-product.

In some embodiments, the present disclosure provides a recombinant microorganism for producing a para-hydroxybenzoic acid (pHBA) or a derivative thereof, said recombinant microorganism comprising a genetically engineered pathway expressing at least one nucleic acid sequence. In some embodiments, the at least one nucleic acid is transfected as a naked nucleic acid or is formulated with one or more agents, such as liposomes. In some embodiments, the nucleic acids is DNA, RNA, cDNA, or combinations thereof, as is appropriate. Additional vectors may include plasmids, viruses, bacteriophages, cosmids, and artificial chromosomes. In a preferred embodiment, the at least one nucleic acid is delivered to the host microorganism using a plasmid, optionally the at least one nucleic acid is delivered as a single synthetic operon. By way of example, transformation of the wild type microorganism (including transduction or transfection) may be achieved by electroporation, ultrasonication, polyethylene glycol-mediated transformation, chemical or natural competence, protoplast transformation, prophage induction, or conjugation. In certain embodiments having active restriction enzyme systems, it may be necessary to methylate a nucleic acid before introduction of the nucleic acid into a microorganism.

In some embodiments, the recombinant microorganism comprises a nucleic acid sequence selected from SEQ ID NOs: 6, 45, 52, 53, 54, 55, 56, 57, 58, 59, 60, 61, 62, 63, 64, 65, 66, 67, or a combination of any two or more thereof. In some embodiments, the recombinant microorganism comprises a nucleic acid sequence set forth in SEQ ID NO: 45. In some embodiments, the recombinant microorganism comprises an exogenous amino acid sequence comprising SEQ ID NOs: 1, 2, 3, 4, 5 or a combination of any two or more thereof.

In some embodiments, the recombinant microorganism comprises a nucleic acid sequence set forth in SEQ ID NO: 6 and a nucleic acid sequence encoding the polypeptide of selected from SEQ ID NO:4; SEQ ID NO: 5; SEQ ID NOs: 1 and 4; SEQ ID NOs: 1 and 5; SEQ ID NOs: 2 and 4; SEQ ID NOs: 2 and 5; SEQ ID NOs: 3 and 4; SEQ ID NOs: 3 and 5; SEQ ID NOs: 1, 3, and 4; SEQ ID NOs: 1, 3, and 5; SEQ ID NOs: 1, 2, and 4 SEQ ID NOs: 1, 2, and 5; SEQ ID NOs: 2, 3, and 4; SEQ ID NOs: 2, 3, and 5; SEQ ID NOs: 1, 2, 3, and 4; or SEQ ID NOs: 1, 2, 3, and 5.

In another aspect, a microorganism is provided that includes a vector comprising a nucleic acid sequence set forth in SEQ ID NOs: 6, 45, 52, 53, 54, 55, 56, 57, 58, 59, 60, 61, 62, 63, 64, 65, 66, 67, or a combination of any two or more thereof. In some embodiments, the microorganism is selected from Methylococcus capsulatus, Methylotuvimicrobia, Methylotuvimicrobium buryatense, Methylomicrobium alcaliphilum, Methylotuvimicrobium album, or Methylobacterium extorquens, and produces pHBA in vivo when grown in a fermentation broth in the presence of a carbon source. In some embodiments, the carbon source comprises methane, methanol, ethanol, carbon monoxide, carbon dioxide, formic acid, or a combination of any two or more thereof.

In some embodiments, a microbial cell factory is constructed by genetically modifying the bacterium Methylomicrobium alcaliphilum 20Z to convert methanol and methane into para-hydroxybenzoic acid, a precursor and feedstock for various industrially relevant chemicals, including aromatic bioplastics. In some embodiments, a genetically engineered microorganism capable of producing at least one chorismate-derived product by fermentation of a carbon source (C1-substrate) is provided.

As described herein, aromatic compounds, such as pHBA, are almost exclusively generated via the shikimate biosynthetic pathway in bacteria, fungi and plants. The shikimate biosynthetic pathway also leads to the production of aromatic amino acids, diverse aromatic precursors, and the biosynthesis of a great variety of secondary metabolites/natural products.

In some embodiments, the naturally occurring microorganism of the present disclosure does not natively produce para-hydroxybenzoic acid. In fact, since ubiquinone is generally only produced in aerobically respiring microorganisms because the chorismate pyruvate lyase is not typically found in methanotropic microorganisms. Although it may be expected that the diversion of chorismate to produce pHBA instead of amino acids would have detrimental effects on the growth or survival of the microorganism, the inventors have shown that the microorganism is not affected to a degree that significantly compromises survival and growth under standard conditions (Examples).

The microorganism may be modified to express or overexpress one or more enzymes that were not expressed or overexpressed in the parental microorganism. Similarly, the microorganism may be modified to contain one or more genes that were not contained by the parental microorganism. In some embodiments, the parental microorganism is S. cerevisiae, Escherichia coli, Corynebacterium glutamicum, Pseudomonas putida, Providencia rustigianii, Bacillus subtilis, Clostridium autoethanogenum, Clostridium ljungdahlii, Clostridium autoethanogenum LZ1561, Clostridium ragsdalei, Listeria monocytogenes, Streptomyces coelicolor, Propionibacterium freudenreichii, Propionibacterium shermanii, Cronobacter sakazakii, Methylococcus capsulatus, Methylotuvimicrobium buryatense, Methylobacterium extorquens, Methylobacterium alcahphilum, or Methylotuvimicrobium album. In a preferred embodiment, the parental microorganism is, Methylococcus capsulatus, Methylotuvimicrobium buryatense, Methylobacterium extorquens, Methylotuvimicrobium album, or Methylobacterium alcahphilum. In some embodiments, the parental microorganism is Methylobacterium alcahphilum 20Z.

As used herein, the term “Overexpressed” means increasing the expression of a nucleic acid or protein in a cell or a microorganism compared to the expression level of the nucleic acid or protein in a wild-type or parental cell or microorganism from which the cell or microorganism is derived. In some embodiments, overexpression may be achieved by any means known in the art, including modifying gene copy number, gene transcription rate, gene translation rate, or enzyme degradation rate.

In some embodiments, the microorganism may be genetically engineered to produce pHBA at a certain selectivity or at a minimum selectivity.

In one embodiment, para-hydroxybenzoic acid production accounts for at least about 5%, 10%, 15%, 20%, 30%, 50%, or 75% of all fermentation products produced by the recombinant microorganism. In one embodiment, derivatives of para-hydroxybenzoic acid account for at least about 5%, 10%, 15%, 20%, 30%, 50%, or 75% of all fermentation products produced by the recombinant microorganism. In one embodiment, derivatives of para-hydroxybenzoic acid account for at least about 5%, 10%, 15%, 20%, 30%, 50%, or 75% of all fermentation products produced by the recombinant microorganism. In one embodiment, chorismate-derived products account for at least about 5%, 10%, 15%, 20%, 30%, 50%, or 75% of all fermentation products produced by the recombinant microorganism.

In one embodiment, the pHBA production accounts for at least 10% of all fermentation products produced by the microorganism, such that the microorganism of the invention has a selectivity for the target product of at least 10%. In another embodiment, the pHBA production accounts for at least 30% of all fermentation products produced by the microorganism of the invention, such that the microorganism has a selectivity for the target product of at least 30%.

In some embodiments, the present disclosure provides a biomass comprising the recombinant microorganism as described herein. In a specific embodiment, the present disclosure provides a biomass comprising a recombinant microorganism, wherein the recombinant microorganism comprises an exogenous nucleic acid encoding a shikimate biosynthesis enzyme and wherein the recombinant microorganism is capable of converting a natural gas-derived feedstock (e.g. methane or methanol) into para-hydroxybenzoic acid or derivatives thereof. Biomass may include a natural product containing hydrolyzable polysaccharides that provide fermentable sugars including any sugars and starch derived from natural resources such as corn, cane, wheat, cellulosic or lignocellulosic material and materials comprising cellulose, hemicellulose, lignin, starch, oligosaccharides, disaccharides and/or monosaccharides, and mixtures thereof.

In some embodiments of the present disclosure, the recombinant microorganism comprises a genetically engineered pathway expressing that is encoded by a single vector, optionally driven by a single promoter.

Typical vectors contain transcription and translation terminators, initiation sequences, and promoters useful for regulation of the expression of the desired nucleic acid sequence. The vectors of the present disclosure may also be used for nucleic acid standard gene delivery protocols. Further, the vector may be provided to the microorganism in the form of a viral vector. Viral vector technology is well known in the art and is described, for example, in Sambrook et al., 4th Edition, Molecular Cloning: A Laboratory Manual, Cold Spring Harbor Laboratory, New York, 2012; and in other virology and molecular biology manuals. Viruses, which are useful as vectors include, but are not limited to, retroviruses, adenoviruses, adeno associated viruses, herpes viruses, Sindbis virus, gamma-retrovirus and lentiviruses.

In some embodiments, the vector comprises at least two, at least three, at least four, or at least five nucleic sequences each encoding a polypeptide selected from an exogenous chorismate pyruvate lyase of EC 5.4.4.2 or EC 4.1.3.40; an exogenous 3-deoxy-D-arabino-heptulosonate-7-phosphate (DAHP) synthase of EC 4.1.2.15, or EC 2.5.1.54; an exogenous shikimate kinase of EC 2.7.1.71; or an exogenous 3-dehydroquinate dehydratase (DHQ dehydratase) of EC 4.2.1.10. In some embodiments, the vector comprises at least two, at least three, at least four, or at least five nucleic sequences each encoding a polypeptide selected from SEQ ID NO: 1, 2, 3, 4, or 5. In some embodiments, the vector comprises at least two, at least three, at least four, or at least five nucleic sequences each encoding a polypeptide selected from P. rustigianii UbiC, C. sakazakii UbiC, E. coli AroG, E. coli AroL, or E. coli AroD.

In some embodiments, the genetically engineered pathway is encoded by a single vector and the single vector comprises: pmxaF-ubiC; pmxaF-ubiC-aroG; pmxaF-ubiC-aroL; pmxaF-ubiC-aroD; pmxaF-ubiC-aroG-aroL; pmxaF-ubiC-aroG-aroD; pmxaF-ubiC-aroL-aroD; or pmxaF-ubiC-aroG-aroL-aroD. In some embodiments, the single vector comprises a nucleic acid sequence selected from SEQ ID NOs: 6, 45, 52, 53, 54, 55, 56, 57, 58, 59, 60, 61, 62, 63, 64, 65, 66, 67, or a combination of any two or more thereof. In some embodiments, the single vector comprises a nucleic acid sequence set forth in SEQ ID NO: 45. In some embodiments, the gene-sequences and regulatory-elements are compiled into a single synthetic operon. In some embodiments, the single synthetic operon comprises a small broad-host-range plasmid, pBBR1MCS or pCM66T, as avector-backbone.

In some embodiments, the single vector further comprises a spacer sequence between each nucleic acid encoding a polypeptide. In some embodiments, the space is encoded by a nucleotide sequence selected from SEQ ID NO: 13, 14, 15, 16, 17, 18, or 19. In some embodiments, the nucleotide sequence of the spacer is

(SEQ ID NO: 12)
CTCGGATACCCTTACTCTGTTGAAAACGAATAGATAGGTT;
(SEQ ID NO: 13)
AAGGAACGGTTATTTCTGCGTAGATCTATCTTACACAGCA;
(SEQ ID NO: 14)
AGGCAACTGAAACGATTCGGATCCTGTATTACTATTCTTA;
(SEQ ID NO: 15)
ACTTTATCTGAGAATAGTCAATCTTCGGAAATCCCAGGTG;
(SEQ ID NO: 16)
TAAAAGTCTCGTAAAGCGTTCTATCAATAACCCGTTGGTG;
(SEQ ID NO: 17)
CCGTCTCAGAATCGGCCGTGAACAATAAAATAGTTTCGGT.

In one aspect, the present disclosure provide a vector comprising a nucleic acid sequence set forth in SEQ ID NOs: 6, 45, 52, 53, 54, 55, 56, 57, 58, 59, 60, 61, 62, 63, 64, 65, 66, 67, or a combination of any two or more thereof. In some embodiments, the present disclosure provide a microorganism comprising a nucleic acid sequence set forth in SEQ ID NOs: 6, 45, 52, 53, 54, 55, 56, 57, 58, 59, 60, 61, 62, 63, 64, 65, 66, 67, or a combination of any two or more thereof. In some embodiments, the microorganism produces para-hydroxybenzoic acid (pHBA) in vivo when grown in a fermentation broth in the presence of a carbon source.

In some embodiments, the present disclosure provides a method of producing para-hydroxybenzoic acid (pHBA) or a derivative thereof, the method comprising: culturing a recombinant microorganism expressing a vector comprising a nucleic acid sequence set forth in SEQ ID NOs: 6, 45, 52, 53, 54, 55, 56, 57, 58, 59, 60, 61, 62, 63, 64, 65, 66, 67, or a combination of any two or more thereof in a fermentation broth; adding a carbon source to the fermentation broth; and isolating the pHBA from the fermentation broth.

A promoter increases or otherwise controls the expression of a particular nucleic acid. In some embodiments, the genetically engineered pathway of the present disclosure is encoded by a single vector, which is driven by a constitutive or inducible promoter. As used herein, a “constitutive promoter” is a nucleotide sequence which, when operably linked with a polynucleotide which encodes or specifies a gene product, causes the gene product to be produced in a cell under most or all physiological conditions of the cell. As used herein, an “inducible promoter” is a nucleotide sequence which, when operably linked with a polynucleotide which encodes or specifies a gene product, causes the gene product to be produced in a cell substantially only when an inducer which corresponds to the promoter is present in the cell.

A “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner. In some embodiments, the promotor is selected from a M. extorquens methanol dehydrogenase promoter (PmxaF), an E. coli (PBAD) ribulokinase promoter (araBp), an E. coli (Plac) β-galactosidase promoter (lacZp), a bacteriophage lambda promoter (λPL), a Wood-Ljungdahl pathway promoter, a ferredoxin promoter, a pyruvate:ferredoxin oxidoreductase promoter, an Rnf complex operon promoter, an ATP synthase operon promoter, or a phosphotransacetylase/acetate kinase operon promoter. In some embodiments, the promotor is encoded by a nucleic acid sequence set forth in SEQ ID NO: 6. In some embodiments, the promotor is a M. extorquens methanol dehydrogenase promoter (PmxaF)

As used herein, the term “Under transcriptional control” or “Operatively linked” means that the promoter is in the correct location and orientation in relation to a polynucleotide to control the initiation of transcription by RNA polymerase and expression of the polynucleotide. As used herein, the term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

In some embodiments, the vector is designed to comprise transcription and translation terminators, initiation sequences, and promoters useful for regulation of the expression of the desired nucleic acid sequence. In some embodiments, the transcription and translation terminor is rrnB T1 (e.g. SEQ ID NO: 43) and T7Te (e.g. SEQ ID NO: 44) sequences. In some embodiments, the single vector encoding the genetically engineered pathway comprises a double-terminator comprising rrnB T1 (e.g. SEQ ID NO: 43) and T7Te (e.g. SEQ ID NO: 44) sequences. In some embodiments, the gene-sequences and regulatory-elements are compiled into a single synthetic operon. In some embodiments, the single synthetic operon comprises the small broad-host-range plasmid pBBR1MCS as vector-backbone.

In some embodiments, the transcription and translation initiation sequence is a ribosomal binding site. In some embodiments, each nucleic acid encoding a polypeptide is conjugated or operably linked to a nucleic acid sequence encoding a ribosomal binding protein; and/or a tag protein. In some embodiments, the nucleotide sequence of the RBS is selected from SEQ ID NO: 7, 8, 9, 10, 11, or 12.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Rationales for Strain-Design and Genetic Engineering

It is unknown whether in type I methanotrophs (e.g. methanotrophiles) like Methylotuvimicrobia the first step (DAHP synthase) of the shikimate pathway is feedback-inhibition regulated, like in many other species (e.g., S. cerevisiae, E. coli, C. glutamicum, P. putida, B. subtilis) that have been studied as hosts for production of aromatics. Averesch and Kromer, Front. Bioeng. Biotechnol., 6, 32 (2018). It does, however, seem natural. Therefore, the present inventors chose to over-express a highly feedback-inhibition resistant mutant-enzyme. Ger et al., Journal of Biochemistry, 116 (5): 986-990 (1994), Purwanto et al., J. of Biotechnology 282: 92-100 (2018).

In a S. cerevisiae study, over-expression of a heterologous shikimate kinase from E. coli showed a significant impact on increased flux to downstream products, but it was not entirely clear why. Rodriguez et al., Metabolic Engineering 31: 181-188 (2015). When considering thermodynamics, this appears, however, logical because this step has a ΔrG° higher than −5.7 kJ/mol (the threshold where the forward-flux is approximately ten-times the reverse-flux). This step has a limited Flux-Force Efficacy. Noor et al., PLOS Computational Biology 10(2): e1003483. Therefore, the only option to increase the forward flux is to increase the total reaction rate by increasing the abundance of the catalyst, through enzyme over-expression. When conducting this analysis for at all the reactions in the pathway, AroD and AroL were identified as being rate-limiting and increasing the total reaction rate appeared essential to increase overall flux. While perviously overlooked, the DHQ dehydratase was also identified as a similarly crucial target as the shikimate kinase. All three enzymes where therefore determined to be critical candidates for the intrinsic self-regulation of the shikimate pathway.

Since para-hydroxybenzoic acid (i.e., pHBA) is a minor metabolite when compared to aromatic amino acids, the natural chorismate lyase is likely not to be very active or only very low expressed or both. As the E. coli enzyme is more or less severely affected by feedback-inhibition, an analogue from a different organism (Providencia rustigianii) was chosen. Providencia rustigianii chorismate lyase is highly feedback-inhibition resistant and also has enhanced activity. Purwanto et al., Journal of Biotechnology 282: 92-100 (2018).

To achieve over-expression, the native Methylotuvimicrobium buryatense methanol dehydrogenase promoter (PmxaF) was chosen, which yields high constitutive expression. Garg et al., Metabolic Engineering 48:175-183 (2018). Genes were codon-optimized, using Methylococcus capsulatus as a proxy, as for that organism an exhaustive codon-usage table is available and the codon-usage appears to be similar. To further maximise translation-rate of the enzymes through enhanced initiation of translation, synthetic ribosomal binding sites were calculated, using the web-based interface of De Novo DNA (salislab.net/software), using Methylobacterium extorquens as a proxy, which was the closest relative organism where data was available. Salis, Methods in Enzymology 498:19-42 (2011).

Accordingly, the genetically engineered pathway of the present disclosure was generated. The genetically engineered pathway was supplemented with a double-terminator comprised of rrnBT1 and T7Te sequences, the gene-sequences and regulatory-elements were compiled into a single synthetic operon, using the small broad-host-range plasmid pBBR1MCS or pCM66T as a vector-backbone. One embodiment of the annotated vector sequence is shown in FIG. 2B and SEQ ID NO. 45. The vector of the present disclosure is stable, functional, and selectable on kanamycin or neomycin. It should be noted that the vectors encoding the genetically engineered pathway of the present disclosure, and the selected combination of genes and regulatory elements have not been reported in a Methylotuvimicrobium context before.

### Example 1: Genetically Engineering the Shikimate Pathway for the Production of Para-Hydroxybenzoic Acid

This example provides the genes, enzymes, nucleotide and amino acid sequences used to make the recombinant microorganisms of the present disclosure. A microbial cell factory was constructed by genetically engineering the bacterium Methylomicrobium alcahphilum 20Z to convert methanol and methane into para-hydroxybenzoic acid (pHBA). A plasmid vector for encoding enzymes of the shikimate pathway for establishing a microbial system for high yield production of para-hydroxybenzoic acid was designed. The plasmid vector comprised four enzymes selected from: (i) AroG^{s180F} (feedback-inhibition resistant Escherichia coli DAHP synthase); (ii) AroD (Escherichia coli 3-dehydroquinate dehydratase); (iii) AroL (Escherichia coli shikimate kinase 2); or (iv) UbiC (Providencia rustigianii chorismate pyruvate-lyase).

### Selected Genes and Enzymes

The genes and enzymes of the genetically engineered pathway of the present disclosures are disclosed in Table 2.

TABLE 2
Selected Genes and Enzymes of the shikimate pathway

| Gene | Source | Enzyme | EC | Charateristics |
|---|---|---|---|---|
| aroG^{S180F}_{Eco} | E. coli | DAHP | [EC 2.5.1.54] | feedback- |
| | | synthase | | inhibition |
| | | | | resistant |
| aroD_{Eco} | E. coli | DHQ | [EC 4.2.1.10] | ΔᵣG′^{m} −5.3 ± |
| | | dehydratase | | 4.1 [kJ/mol] |
| aroL_{Eco} | E. coli | shikimate | [EC 2.7.1.71] | ΔᵣG′^{m} −5.2 ± |
| | | kinase | | 9.2 [kJ/mol] |
| ubiC_{Pru} | P. | chorismate | [EC 4.1.3.40] | highest activity |
| | rustigianii | lyase | | |

The amino acid sequences of the polypeptides encoded by the genes disclosed in Table 2, and are shown below.

AroG_{Eco}^{S180F} (feedback-inhibition resistant
Escherichia coli DAHP synthase)
(SEQ ID NO: 1)
MNYQNDDLRIKEIKELLPPVALLEKFPATENAANTVAHARKAIHKILKG
NDDRLLVVIGPCSIHDPVAAKEYATRLLALREELKDELEIVMRVYFEKP
RTTVGWKGLINDPHMDNSFQINDGLRIARKLLLDINDSGLPAAGEFLDM
ITPQYLADLMSWGAIGARTTESQVHRELASGLFCPVGFKNGTDGTIKVA
IDAINAAGAPHCFLSVTKWGHSAIVNTSGNGDCHIILRGGKEPNYSAKH
VAEVKEGLNKAGLPAQVMIDFSHANSSKQFKKQMDVCADVCQQIAGGEK
AIIGVMVESHLVEGNQSLESGEPLAYGKSITDACIGWEDTDALLRQLAN
AVKARRG
AroD_{Eco} (Escherichia coli 3-dehydroquinate
dehydratase)
(SEQ ID NO: 2)
MKTVTVKDLVIGTGAPKIIVSLMAKDIASVKSEALAYREADFDILEWRV
DHYADLSNVESVMAAAKILRETMPEKPLLFTFRSAKEGGEQAISTEAYI
ALNRAAIDSGLVDMIDLELFTGDDQVKETVAYAHAHDVKVVMSNHDFHK
TPEAEEIIARLRKMQSFDADIPKIALMPQSTSDVLTLLAATLEMQEQYA
DRPIITMSMAKTGVISRLAGEVFGSAATFGAVKKASAPGQISVNDLRTV
LTILHQA.
AroL_{Eco} (Escherichia coli shikimate kinase 2)
(SEQ ID NO: 3)
MTQPLFLIGPRGCGKTTVGMALADSLNRRFVDTDQWLQSQLNMTVAEIV
EREEWAGFRARETAALEAVTAPSTVIATGGGIILTEFNRHFMQNNGIVV
YLCAPVSVLVNRLQAAPEEDLRPTLTGKPLSEEVQEVLEERDALYREVA
HIIIDATNEPSQVISEIRSALAQTINC
UbiC_{Pru} (Providencia rustigianii chorismate
pyruvate-lyase)
(SEQ ID NO: 4)
MHETIFTHHPIDWLNEDDESVPNSVLDWLQERGSMTKRFEQHCQKVTVI
PYLERYITPEMLSADEAERLPESQRYWLREVIMYGDNIPWLIGRTLIPE
ETLTNDDKKLVDIGRVPLGRYLFSHDSLTRDYIDIGTSADRWVRRSLLR
LSQKPLLLTEIFLPESPAYR
UbiC_{Csa} (Cronobacter sakazakii chorismate
pyruvate-lyase)
(SEQ ID NO: 5)
MSHPALRQLRALSFFDDISTLDSSLLDWLMLEDSMTRRFEGFCERVTVD
MLFEGFVGPEALEEEGEFLPDEPRYWLREILLCGDGVPWLVGRTLVPES
TLCGPELALQQLGTTPLGRYLFTSSTLTRDFIQPGRSDELWGRRSLLRL
SGKPLLLTELFLPASPLYGEEK

### Promoters

To genetically engineer the shikimate pathway for enhanced production of pHBA in a recombinant microorganism (e.g., for generating the expression of the enzymes), a native Methylotuvimicrobium buryatense 5 GB1C methanol dehydrogenase promoter (PmxaF; SEQ ID NO: 6) was used to engineer a single vector comprising the genetically engineered pathway. Alternatively, a native M. extorquens methanol dehydrogenase promoter was also used. The PmxaF showed high constitutive gene expression. A constitutive methanol dehydrogenase promoter (PmxaF) was used for expression of the genes disclosed in Table 2 in Methylomicrobium alcahphilum.

The nucleic acid sequence of the PmxaF promoter used in the present disclosure is disclosed below:

(SEQ ID NO: 6)
aattaaaccgggaatgatgtcggatatttaacggcaaagccatgggagc
ttttcccgaatttgaatgccgacatactctcgggatattttccctgttt
tttcttagcgcttttcccgtcatctgggtgctgtattccgtaacgtcgc
atcccgctccttccgtatgattaccgtccgtgcgctgccctctatgaat
gattcgttatgcgccttgatcaagctaagccggttgtaacaacaaacac
cgcaatcaatagggggccgcgccgacattatgcgaaaaatcaatctgga
ggaattATG[. . .]TAA

The PmxaF promoter is tightly repressed when lanthanide metals are present (e.g. 30 μM lanthanum). Groom et al., J. Bacteriol. 201(15):e00120-19 (2019). In contrast, the MxaFI promoter contains calcium in its active site, while XoxF promoter contains a lanthanide. The lanthanide-mediated methanol dehydrogenase switch is regulated by MxaY. Chu et al., PeerJ 4:e2435 (2016). Because lanthanide metals contaminate a lot of glassware, it is necessary to soak glassware with 1 M HCl (overnight), rinse with DI water, and then autoclave to remove any residual lanthanide metals contaminant.

Furthermore, the genes described in Table 2 were codon-optimized. To codon optimize the heterologous sequences for expression in M. alcahphilum, Methylococcus capsulatus was used as proxy for codon-optimisation. M. capsulatus was selected because an exhaustive review of a codon-usage table showed that the codon-usage appeared similar to that of M. alcahphilum.

### Ribosomal Binding Site (RBS)

To maximize translation-rate of the polypeptides (e.g., enzymes of the shikimate pathway for heterologous expression) of the present disclosure, initiation of translation was enhanced using synthetic ribosomal binding sites (RBS; SEQ ID NOs: 7-12). These synthetic ribosomal binding sites were also optimized for expression in M. alcaliphilum using a web based interface of De Novo DNA. Salis et al., Methods Enzymol. 498:19-42 (2011). Methylobacterium extorquens, which is the closest relative organism for which data is available was used as a proxy to optimize the synthetic ribosomal binding sites. Methylomicrobium album was used as a proxy to optimize the ribosomal binding site (RBS) and the operon design.

The codon-optimized sequences for the Ribosomal Binding Site (RBS) of each gene and promoter is shown below.

TCTGGAGGAATT (PmxaF)
TTTAAGAAGGAGATATACAT (PBAD)
GCCGTAGTACCGGCCCAATACAGTACTTTTTTT (ubiC)
CACCAAACGAGAAGAACTCAGACTTTTTT (aroG)
ACCATCTCAAGAGAACTGGCAAGTTCTCGCACTTTTTTT (aroL)
AAAACTACGCTCGAGAACGAGTATTATTTTTTG (aroD)

### Terminator

In addition, the single vector encoding the genetically engineered pathway was supplemented with a double-terminator comprised of rrnB T1 (e.g. SEQ ID NO: 43) and T7Te (e.g. SEQ ID NO: 44) sequences (as found on pBADTrfp). The gene-sequences and regulatory-elements were compiled into a single synthetic operon, using the small broad-host-range plasmid pBBR1MCS as vector-backbone.

L3S2P51
CTCGGTACCAAAAAAAAAAAAAAAGACGCTGAAAAGCGTCTTTTTTCGT
TTTGGTCC
rrnB T2-T3Te:
AGAAGGCCATCCTGACGGATGGCCTTT-ggctcaccttcacgggtgggc
ctttcttcg

### Spacers

The nucleotide sequences for the spacers that can be used with the present invention include, but are not limited to, the following:

CTCGGATACCCTTACTCTGTTGAAAACGAATAGATAGGTT
AAGGAACGGTTATTTCTGCGTAGATCTATCTTACACAGCA
AGGCAACTGAAACGATTCGGATCCTGTATTACTATTCTTA
ACTTTATCTGAGAATAGTCAATCTTCGGAAATCCCAGGTG
TAAAAGTCTCGTAAAGCGTTCTATCAATAACCCGTTGGTG
CCGTCTCAGAATCGGCCGTGAACAATAAAATAGTTTCGGT
ATTATTGACCACTTCCGAGTAGAATCGTGCTTCAGTAAGA

### Lumio and 6×His Tag

Table 3 provides the nucleotide and amino acid sequence of the Lumio tag used and that can be used in the single vector encoding the genetically of the present disclosure.

TABLE 3
Lumio Tag

| Spacer | Tetra-Cystein | Spacer |
|---|---|---|
| Gly Ser Gly Ser | Cys Cys Pro Gly Cys Cys | Gly Gly |
| GGC TCC GGC TCC | TGC TGC CCG GGC TGC TGC | GGT GGT |
| GGG TCG GGG TCG | TGT TGT CCC GGG TGT TGT | GGC GGC |
| GGC TCG GGC TCG | TGC TGT CCG GGG TGC TGT | GGG GGG |
| GGG TCC GGG TCG | TGT TGC CCC GGC TGT TGC | |

The nucleotide sequence for the 6×His tag is: CATCACCATCACCATCAC; or CACCATCACCATCACCAT

Single Vector Operon for Methylococcus capsulatus Expression

The gene-sequences and regulatory-elements were compiled into a single synthetic operon, using the small broad-host-range plasmid pBBR1MCS as vector-backbone. The annotated vector sequence is set forth in SEQ ID NO: 45 (gBlock (genes codon-optimised for Methylococcus capsulatus Bath). This single vector encoding the genetically engineered pathway of the present disclosure is novel because to the present inventor's knowledge, the vectors of the present disclosure have not been reported in a Methylotuvimicrobium context before.

The annotated nucleotide sequence of the single operon vector is set forth in SEQ ID NO: 45 and shown below.

ggcgccccagctggcaattccaattaaaccgggaatgatgtcggatatt
taacggcaaagccatgggagcttttcccgaatttgaatgccgacatact
ctcgggatattttccctgttttttcttagcgcttttcccgtcatctggg
tgctgtattccgtaacgtcgcatcccgctccttccgtatgattaccgtc
cgtgcgctgccctctatgaatgattcgttatgcgccttgatcaagctaa
gccggttgtaacaacaaacaccgcaatcaatagggggccgcgccgacat
tatgcgaaaaatcaatctggaggaattGCCGTAGTACCGGCCCAATACA
GTACTTTTTTTATGCATGAAACCATCTTCACGCACCATCCGATTGACTG
GTTGAACGAAGACGACGAGAGCGTCCCCAACTCCGTGCTGGATTGGCTG
CAGGAACGCGGTTCCATGACGAAACGTTTCGAACAGCATTGCCAAAAGG
TCACGGTCATCCCGTACCTGGAGCGCTACATCACGCCCGAGATGCTCTC
GGCGGACGAGGCGGAACGCCTGCCGGAATCCCAACGCTATTGGCTCCGC
GAGGTCATCATGTATGGCGATAACATCCCGTGGCTGATCGGACGCACGC
TGATCCCGGAAGAGACGCTGACCAACGATGACAAAAAGCTGGTGGACAT
CGGTCGGGTGCCGTTGGGCCGTTATCTGTTCTCCCACGACTCGTTGACC
CGCGATTACATCGATATCGGCACCAGCGCCGACCGCTGGGTCCGGCGGT
CGTTGCTGCGGCTGAGCCAGAAGCCCCTGCTGCTGACGGAAATCTTTCT
GCCGGAATCCCCCGCCTATCGCTAATGCTGCCCGGGCTGCTGCTAACAC
CAAACGAGAAGAACTCAGACTTTTTTATGAACTACCAGAACGATGACCT
GCGGATTAAGGAAATCAAGGAGTTGCTGCCGCCCGTCGCCCTGCTGGAG
AAATTCCCGGCCACCGAAAACGCGGCCAACACCGTCGCCCATGCCCGCA
AAGCGATCCACAAGATCCTGAAGGGCAACGATGACCGTTTGTTGGTCGT
GATCGGCCCCTGCAGCATTCACGATCCGGTCGCCGCGAAAGAATACGCC
ACCCGTTTGCTGGCGTTGCGGGAGGAACTCAAGGATGAGTTGGAAATCG
TCATGCGTGTGTACTTTGAAAAACCGCGGACCACCGTGGGCTGGAAGGG
TTTGATTAATGACCCGCACATGGATAACAGCTTCCAGATCAACGACGGT
CTGCGTATCGCGCGGAAATTGCTCCTGGACATCAACGACAGCGGATTGC
CCGCGGCCGGCGAATTTTTGGACATGATCACCCCGCAATACCTGGCCGA
CCTGATGTCGTGGGGTGCCATCGGCGCCCGCACGACCGAATCCCAGGTC
CACCGCGAACTCGCCAGCGGTCTGTTCTGTCCGGTCGGTTTCAAAAACG
GGACCGACGGGACGATCAAGGTGGCCATCGACGCGATCAATGCGGCCGG
AGCCCCCCACTGCTTCCTGAGCGTCACCAAGTGGGGTCATAGCGCCATC
GTCAACACGTCCGGCAACGGCGATTGCCATATCATCCTGCGGGGCGGTA
AGGAGCCCAACTACAGCGCCAAGCATGTCGCCGAAGTCAAGGAAGGGCT
CAACAAGGCCGGACTGCCGGCCCAGGTGATGATCGACTTTAGCCACGCC
AATTCGAGCAAGCAGTTCAAGAAACAAATGGATGTGTGCGCGGACGTCT
GTCAACAGATCGCGGGTGGTGAAAAGGCCATCATCGGTGTGATGGTCGA
AAGCCACCTGGTGGAAGGCAACCAGTCCCTCGAATCCGGCGAGCCCCTG
GCCTACGGAAAATCGATCACCGACGCGTGCATCGGGTGGGAGGATACGG
ATGCCCTGTTGCGTCAGCTGGCCAATGCGGTCAAGGCCCGGCGCGGTTA
ATGTTGTCCCGGGTGTTGTTAAACCATCTCAAGAGAACTGGCAAGTTCT
CGCACTTTTTTTATGACCCAGCCCCTGTTTCTGATCGGCCCCCGTGGTT
GTGGAAAGACGACGGTCGGGATGGCGCTGGCCGACAGCCTGAATCGCCG
TTTCGTCGACACGGATCAGTGGCTGCAGTCGCAGCTGAACATGACGGTG
GCGGAAATCGTGGAACGGGAAGAATGGGCCGGCTTTCGCGCCCGGGAGA
CCGCCGCCCTGGAAGCGGTCACCGCCCCGAGCACGGTCATTGCCACCGG
CGGTGGCATCATCCTGACCGAATTTAACCGCCATTTCATGCAGAATAAT
GGTATCGTGGTCTACCTGTGTGCCCCGGTGTCGGTCTTGGTGAATCGCC
TCCAGGCGGCCCCCGAGGAAGACTTGCGTCCGACCTTGACGGGCAAACC
CCTGTCGGAGGAAGTGCAGGAAGTCCTGGAGGAACGGGATGCCTTGTAC
CGGGAAGTGGCCCACATCATCATCGACGCCACCAACGAGCCGTCGCAGG
TGATCTCGGAAATCCGTAGCGCCCTGGCCCAGACCATCAACTGCTAATG
CTGTCCGGGGTGCTGTTAAAAAACTACGCTCGAGAACGAGTATTATTTT
TTGATGAAAACCGTCACGGTCAAAGATTTGGTGATTGGTACGGGTGCGC
CCAAAATCATCGTCTCCCTGATGGCGAAAGACATCGCGAGCGTGAAGAG
CGAAGCGTTGGCGTACCGGGAAGCGGACTTCGATATCTTGGAATGGCGC
GTGGACCACTACGCCGACCTGTCGAACGTGGAATCCGTGATGGCCGCCG
CGAAGATTTTGCGCGAGACCATGCCGGAGAAGCCCTTGCTGTTTACCTT
CCGTTCGGCCAAGGAAGGCGGCGAGCAGGCCATTTCGACCGAGGCCTAT
ATCGCCCTCAACCGCGCCGCCATCGATTCCGGCCTCGTGGACATGATCG
ACTTGGAACTGTTCACGGGCGATGACCAAGTCAAGGAAACCGTCGCCTA
CGCCCACGCCCACGACGTGAAAGTGGTCATGTCGAACCACGACTTCCAT
AAGACGCCGGAAGCCGAGGAAATCATCGCGCGCCTGCGTAAGATGCAGT
CGTTCGATGCCGATATTCCCAAGATTGCCCTGATGCCGCAGTCCACGTC
CGACGTCCTGACGCTGCTGGCCGCCACGCTGGAGATGCAGGAACAGTAT
GCGGACCGCCCGATCATCACGATGAGCATGGCCAAGACGGGAGTGATTA
GCCGTTTGGCGGGCGAAGTGTTCGGCAGCGCGGCCACGTTTGGGGCGGT
GAAGAAAGCCTCCGCGCCGGGCCAGATTAGCGTGAATGACTTGCGCACC
GTCCTGACCATTTTGCACCAGGCGTAATGTTGCCCCGGCTGTTGCTAAg
gatctccaggcatcaaa

### Additional Construct Vector-Set Based on

Additional vector sets include, but are not limited to: pBBR1pmxaF-ubiC-aroG-aroL-aroD; pBBR1pmxaF-ubiC-aroG-aroL; pBBR1pmxaF-ubiC-aroG; pBBR1pmxaF-ubiC; pCM66TpmxaF-ubiC-aroG-aroL-aroD; pCM66TpmxaF-ubiC-aroG-aroL; pCM66TpmxaF-ubiC-aroG; and/or pCM66TpmxaF-ubiC. The pBBR1 orpCM66T backbone is about 3963 nucleotides (bp) and the pHBA cassette is about 3186 nucleotides (bp). Primers for generating the vectors include:

pBBR1_backbone for:
taaggatctccaggcatcaaa Tₘ: 63° C.
pBBR1_backbone rev:
attggaattgccagctgg Tₘ: 63° C.
pHBA_cassette for:
ccagctggcaattccaat Tₘ: 63° C.
pHBA_cassette rev:
tttgatgcctggagatccttatta Tₘ: 63° C.

### Example 2: Materials and Methods for Genetically Engineering the Recombinant Microorganism

Expression of the novel vectors of the present disclosure in a microorganism of the Methylotuvimicrobium species was stable and functional, and selectable using various selective agents (e.g., ampicillin, kanamycin or neomycin). In particular, Methylomicrobium alcahphilum 20Z expressing the genetically engineered pathway of the present disclosure in the absence of methanol on version 2.1 Nitrate Mineral Salts medium in the presence of 50-100 μg/ml of kanamycine and nutrient broth (NMS2.1kan+NB). In some cases, 30 μM of Lanthanum chloride (LaCl3) were added to the growth medium to suppress the PmxaF promoter. Accordingly, the recombinant microorganisms of the present disclosure can growth under all the conditions tested. Moreover, the nucleic acids encoding the polypeptides of the genetically engineered pathway were expressed and functional when expressed in M. alcahphilum. These results were novel and unexpected because M alcahphilum has never been used for the production of pHBa. In one aspect, the present disclosure provides a novel microorganism for the production of pHBa in vivo.

The construction of the plasmid vector (pBBR1pmxaF-ubiC-aroG-aroL-aroD or pCM66TpmxaF-ubiC-aroG-aroL-aroD) or any plasmid of the present disclosure was carried out using GenScript. The plasmid vector (pBBR1pmxaF-ubiC-aroG-aroL-aroD or pCM66TpmxaF-ubiC-aroG-aroL-aroD) was then introduced into the host organism (e.g. Methylomicrobium alcahphilum 20Z). para-hydrozybenzoic acid, or derivatives thereof were produced in liquid cultures (e.g fermentation broth) using methanol and methane as a substrate (e.g. carbon source). Production of para-hydroxy-benzoate or para-hydroxybenzoic acid, or derivative thereof was confirmed by high-performance liquid chromatography.

Additional materials and methods used to make the recombinant microorganism of the present disclosure are shown below.

### Conjugation.

Conjugation of the microbes was performed according to Puri et al., Appl. Environ Microbiol. 81(5):1775-81 (2015). The NMS2.1 mating-plates, which contain less NaCl than the standard medium (2 g/L) and are supplemented with 15% (v/v, ≙1.2 g/L) nutrient broth. The final concentrations of sodium carbonate and phosphate buffer are also adjusted, to 5 mM and 5.8 mM, respectively. For conjugation, Methylomicrobium cells (from liquid pre-culture) are spread onto NMS2.1 mating-plates and grown over-night. An equal volume of E. coli donor-biomass (also from liquid pre-culture) containing the vector of interest is then added to each plate by spreading, and the plate is incubated at 30° C. for another 1-2 days. Biomass (containing exconjugants) is then collected (with a pipette tip) and spread onto NMS2.1 plates containing kanamycin (25 μg/mL) to select for transconjugants. When colonies appear (usually after 5 days) the plate is replica-plated on kanamycin-containing NMS2.1 to reduce background and purify transconjugants from the donor-strain (rifamycin 50 μg/mL may be used for additional selection pressure towards Methylomicrobium). Regrown colonies may be picked and streaked on NMS2.1-kanamycin plates for screening and characterisation.

### Cultivation

M. buryatense 5 GB1 grows over a wide temperature (4-45° C., with 30° C. being optimal), and pH range (6-10, with an optimum between pH 8-8.5). Kalyuzhnaya et al., International J. Systematic Evolutionary Microbiology 58 (3): 591-596 (2008). It requires NaHCO₃/Na₂CO₃, ideally in conc. of 0.1-0.3 M and tolerates salt (NaCl) conc. of 0.2%-8%, with an optimum at 0.75% [w/v]. It endures extremely high methanol conc. up to 7% (v/v), with 1% being optimal.

M. alcaliphilum 20Z^{R} grows well at 25-30° C. in a pH range of 7.2-9.5 (ideally 9-9.5). Does not grow below pH 7 (slowly at pH 7, no growth at pH 6.8). It requires NaHCO₃ or NaCl for growth in alkaline medium (sodium ions at 0.05 M) and tolerates up to 1.5 M NaCl (8.8% [wil]).

### Medium

Modified nitrate mineral salts medium (NMS2.1) contains: 0.2 g/L MgSO₄·7H₂O, 0.02 g/L CaCl₂·6H₂O, 1 g/L KNO₃, and 7.5 g/L NaCl, as well as 1×trace elements. Nguyen et al., Biotechnology for Biofuels 12, art. 147 (2019). 500×trace elements contains: 1 g/L Na₂-EDTA, 2 g/L FeSO₄·7H₂O, 0.8 g/L ZnSO₄·7H₂O, 0.03 g/L MnCl₂·4H₂O, 0.03 g/L H₃BO₃, 0.2 g/L CoCl₂·6H₂O, 0.6 g/L CuCl₂·2H₂O, 0.15 g/L Na₂O₄W·2H₂O, 0.02 g/L NiCl₂·6H₂O, and 0.05 g/L Na₂MoO₄·2H₂O. Final concentrations of 50 mM sodium carbonate buffer (pH8.8-9) and 2.3 mM phosphate buffer (pH 6.8) are added immediately before use. Gaseous phase should be 25% (v/v) methane in air. Methanol at 0.2% (v/v) may serve as alternative carbon-source.

### Quantification (Jorissen's Test)

To determine the formation of pHBA by the recombinant microorganism of the present disclosure, a Jorissen's Test was conducted. The test is based on the partial conversion of benzoic acid to salicylic acid by hydrogen peroxide. Two reagents are needed for the test. Reagent I is 2% sodium nitrite (NaNO₂) solution in water. Reagent II is 0.3% copper sulphate (CuSO₄) solution in 10% acetic acid. “To a 25 mL sample of salicylic acid, 1 mL of each of the reagents (I) and (II) are added, and the mixture is heated to 100° C. for 15 min., then cooled, and diluted to 50 mL. The red colour produced is matched by adding the standard colour solution to a blank consisting of 50 mL of water containing 1 mL of reagent (II). Edwards et al. Analyst 62:172-177 (1937).

As little as 4 mg/L of salicylic acid gives a distinct pink colour. With para-(hydroxybenzoic) acid under the same conditions 40 mg/L (≈0.3 mM) gives a very faint yellow colour, and even when the quantity is increased to 400 mg/L (≈3 mM) of the solution the colour produced is distinctly yellow, not pink. Edwards et al. Analyst 62:178-185 (1937).

### Adapted Protocol

The following steps were used to determine the presence of pHBA in a fermentation broth comprising a cultured recombinant microorganism of the present disclosure.
Step 1: Standards were prepared in 6 serial-dilutions starting with 250 μM (≙7 concentrations and “0”). Samples were diluted 1:10 with water to generate standards in water. Samples were also diluted in NMS (undiluted) to generate standards in NMS.
Step 2: 1:25 2% NaNO₂ solution and 0.3% CuSO₄ solution in 10% acetic were added to the samples and standards. For example, 40 μM of each reagent was used for 1 mL of sample. The reagent were added sequentially and mixed in between each addition.
Step 3: The samples and standards were incubated at 95° C. for 1 h, followed by a 18 h incubation at room temperature.
Step 4: Absorbance was measure at 300 nm for water/1:10 dilution, or @325 nm for NMS/undiluted.

To ensure the effectiveness of the test, two controls were included. Controls of non-producing strain and additional standards of pHBA in supernatant of non-producing strain to eliminate potential overestimation of pHBA-content due to unspecificity of assay.

### Quantification (HPLC).

Analytics were was based on a previously published HPLC-method for detection of organic acids (formic, acetic, lactic, propionic and butyric acid). Lohner et al. ISME Journal 8: 1673-1681 (2014). In short, the procedure was as follows: Samples (1 mL) were filtered (PVDF or PES syringe filters, 0.2 μm pore-size) into HPLC sampling vials. Analysis of 50 μL sample-volume was performed on a Agilent 1260 Infinity HPLC system, using an Aminex HPX-87H column (Bio-Rad, Hercules, CA, USA) with 5-mM H₂SO₄ as the eluent, at a flow rate of 0.7-mL/min. Aliphatic organic acids as well as 4-hydroxybenzoic acid were identified by comparison to standards (ten serial-dilutions, starting with 10 mM as the highest concentration), according to retention time (59 min in case of para-hydroxybenzoic acid) using a variable wavelength detector (210 nm) (55° C.). The lower detection limit for 4-hydroxybenzoic acid was 10 μM.

### Example 3: xylE-Analogous Biosensor

This example provides a novel test for measuring pHBA production in the fermentation broth.

Analogous to the use of catechol 2,3-dioxygenase as reporter to quantitate gene expression(catechol_dioxygenase), where the formation of 2-hydroxymuconate semialdehyde results in a strong yellow colour, the present inventor used the conversion of protocatechuate (PCA) to 3-carboxy-cis,cis-muconate (protocatechuate_3,4 dioxygenase) as a measure for pHBA production in the fermentation broth of the present disclosure. This novel test required the conversion of pHBA to PCA, either in vitro with 4-hydroxybenzoate 3-monooxygenase (NAD(P)H) or an analogous chemical reaction.

A streaks of primary ex-conjugant strains (harbouring pBBR1PmxaF-ubiC-aroG-aroL-aroD or pCM66TpmxaF-ubiC-aroG-aroL-aroD) showed heterogenous colony-morphology/growth-phenotype (poor growth) on solid medium containing 0.2% methanol as carbon-source (i.e., NMS2.1kan+MeOH). Fast growers were mutants with changed 4HBA-operon sequence. Fast-growing mutant #1 produced some 4HBA. The hypothesis for these mutants is that the methanol-dehydrogenase promoter became highly active in the presence of external methanol, leading to enzyme and/or end-product toxicity. The toxic conditions in turn favoured re-arrangement and/or point mutations of the plasmid. Streaks of the transconjugants on solid medium without methanol, grew comparatively well The solid medium was supplemented with nutrient broth, NMS2.1kan and MeOH. Toxicity tests with varying 4HBA concentrations was performed.

A 4HBA toxicity tests was performed and showed streaks of the background-strains on plates with horizontal 4HBA-gradient (0-10 mM). These streaks revealed a significant inhibitory effect of 4HBA on growth at pH 8.9. In addition, the M. buryatense 5GB1 (5G) strain was more severely affected than the M. alcahphilum 20Z strain.

pHBA (i.e.,4HBA) production was also determined. To screen for pHBA production the effect of methanol and methane on the M. buryatense 5GB1 (5G) strain and the M. alcahphilum 20Z strain at two different pH (e.g., pH 8.9 vs. pH 7.3). It was hypothesized that toxicity and/or selection pressure would be lower in liquid medium, due to more dilute cell-density. Accordingly, precultures were started on NMS2.1 without methanol using 2 g/L NB and only 25 mg/L Kanamycin (Kan). Growth appeared after ≈1 week of incubating with “cap-on”. Cultures were then spiked with 0.5 mL/L methanol and caps were removed to allow oxygen-transfer. After 24 h, the medium (i.e. fermentation broth) was exchanged. Growth was measured with sampling every about 24 h. Substrate (carbon source or methanol, methane) were added to the new growth medium (fermentation broth). This was repeated twice every 48 h until a sufficient amount of cells was obtained.

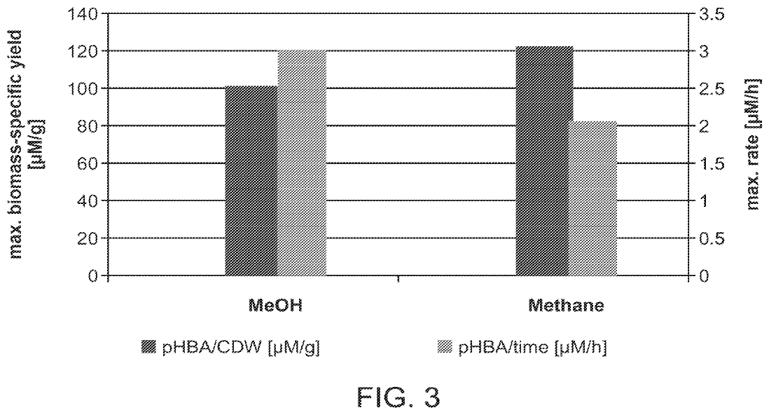

From methanol 0.145 mM pHBA were obtained after 48 h with the recombinant M. alcahphilum 20Z strain (20Z*) expressing ubiC_{Pru} aroG_{Eco}^{S180F} aroL^{Eco} aroD_{Eco}. Past 48 h the pHBA concentration surprisingly dropped to 0. From methane 0.203 mM pHBA were obtained after 120 h. an even higher titer of pHBA was surprisingly obtained when the recombinant microorganism was grown on complex medium containing methanol (MeOH) (FIG. 3). The surprisingly higher productivity from methanol was likely due to better mass-transfer. Nonetheless, the higher titer and per-biomass yield from methane indicated that methane (CH₄) was a better (i.e., more efficient) carbon source and/or energy source for the production of pHBA.

Additional clones of the recombinant M. alcahphilum 20Z strain (20Z*) expressing a plasmid vector encoding ubiC_{Pru} aroG_{Eco}^{S180F} aroL_{Eco} aroD_{Eco} were produced, which showed homogenous growth. Transconjugants of the M. buryatense 5GB1C (5G) expressing FubiC_{Pru} aroG_{Eco}^{S180F} aroL_{Eco} aroD_{Eco}, were also obtained. The M. buryatense 5GB1C (5G) recombinant strains also grew homogeneously. However, the M. buryatense 5GB1C (5G) grew poorly on NMS2.1 and methanol (MeOH), but grew well on well on NMS2.1 and NB. The the recombinant M. alcahphilum 20Z (20Z*) based strains did not show this behavior. As such, the the M. buryatense 5GB1C (5G) recombinant strain may be better aromatics producers, but they have a lower toxicity threshold.

### Example 4: Construction of pHBA Vector Using RK2/RP4-Plasmid

An alternative vector encoding a genetically engineered shikimate pathway that was resistant to genetic rearrangement was generated. The improved vector (e.g. pHBA cassette) contained a pCM66T backbone, which is a change of vector class (pBBR1MCS to pCM66T. The improved and stable vector comprised pCM66Tp_{mxaF}ubiC_{Pru}-aroG_{Eco}^{S180F}-aroL_{Eco}-aroD_{Eco} (SEQ ID NO: 69). The primers used to generate the improved alternative vector for expressing the shikimate pathway is shown below.

Primers used to generate the new vector include:

Kan_fwd
(SEQ ID NO: 70)
gcaccatgttggaatttaatcgc
Kan_rev
(SEQ ID NO: 71)
gcgattaaartccaacatggatgc
PmxcaF for
(SEQ ID NO: 72)
aattaaaccgggaatgatgt
db1-term rev
(SEQ ID NO: 73)
cgttttatttgatgcctgga
db1-term rev
(SEQ ID NO: 74)
ctccaggcatcaaataaaacgaaagg
PmxcaF-pCM66T rev
(SEQ ID NO: 75)
cattcccggtttaattattgcgttgcgctcac
ubiC_seq for
(SEQ ID NO: 76)
CTCTCGGCCGACGAAGCCGAAC
ubi_C_seq rev
(SEQ ID NO: 77)
CCGATCAGCCAGGGGATGTIATCG

TABLE 1
Sequences

| SEQ ID NO: | Gene name | Description | Sequence |
|---|---|---|---|
| 1 | AroG_{Eco}^{S180F} | Escherichia coli | MNYQNDDLRIKEIKELLPPVALLEKFPATE |
| | | feedback-inhibition | NAANTVAHARKAIHKILKGNDDRLLVVIG |
| | | resistant DAHP | PCSIHDPVAAKEYATRLLALREELKDELEI |
| | | synthase | VMRVYFEKPRTTVGWKGLINDPHMDNSF |
| | | | QINDGLRIARKLLLDINDSGLPAAGEFLDM |
| | | | ITPQYLADLMSWGAIGARTTESQVHRELA |
| | | | SGLFCPVGFKNGTDGTIKVAIDAINAAGAP |
| | | | HCFLSVTKWGHSAIVNTSGNGDCHIILRG |
| | | | GKEPNYSAKHVAEVKEGLNKAGLPAQVM |
| | | | IDFSHANSSKQFKKQMDVCADVCQQIAG |
| | | | GEKAIIGVMVESHLVEGNQSLESGEPLAY |
| | | | GKSITDACIGWEDTDALLRQLANAVKARRG |
| 2 | AroD_{Eco} | Escherichia coli 3- | MKTVTVKDLVIGTGAPKIIVSLMAKDIAS |
| | | dehydroquinate | VKSEALAYREADFDILEWRVDHYADLSN |
| | | dehydratase | VESVMAAAKILRETMPEKPLLFTFRSAKE |
| | | | GGEQAISTEAYIALNRAAIDSGLVDMIDLE |
| | | | LFTGDDQVKETVAYAHAHDVKVVMSNH |
| | | | DFHKTPEAEEIIARLRKMQSFDADIPKIAL |
| | | | MPQSTSDVLTLLAATLEMQEQYADRPIIT |
| | | | MSMAKTGVISRLAGEVFGSAATFGAVKK |
| | | | ASAPGQISVNDLRTVLTILHQA |
| 3 | AroL_{Eco} | Escherichia coli | MTQPLFLIGPRGCGKTTVGMALADSLNRR |
| | | shikimate kinase 2 | FVDTDQWLQSQLNMTVAEIVEREEWAGF |
| | | | RARETAALEAVTAPSTVIATGGGIILTEFN |
| | | | RHFMQNNGIVVYLCAPVSVLVNRLQAAP |
| | | | EEDLRPTLTGKPLSEEVQEVLEERDALYRE |
| | | | VAHIIIDATNEPSQVISEIRSALAQTINC |
| 4 | UbiC_{Pru} | Providencia | MHETIFTHHPIDWLNEDDESVPNSVLDWL |
| | | rustigianii chorismate | QERGSMTKRFEQHCQKVTVIPYLERYITPE |
| | | pyruvate-lyase | MLSADEAERLPESQRYWLREVIMYGDNIP |
| | | | WLIGRTLIPEETLTNDDKKLVDIGRVPLGR |
| | | | YLFSHDSLTRDYIDIGTSADRWVRRSLLRL |
| | | | SQKPLLLTEIFLPESPAYR |
| 5 | UbiC_{CROS8} | Cronobacter sakazakii | MSHPALRQLRALSFFDDISTLDSSLLDWL |
| | | chorismate pyruvate- | MLEDSMTRRFEGFCERVTVDMLFEGFVGP |
| | | lyase | EALEEEGEFLPDEPRYWLREILLCGDGVP |
| | | | WLVGRTLVPESTLCGPELALQQLGTTPLG |
| | | | RYLFTSSTLTRDFIQPGRSDELWGRRSLLR |
| | | | LSGKPLLLTELFLPASPLYGEEK |
| 6 | P_{mxaF} | M. extorquens | aattaaaccgggaatgatgtcggatattta |
| | | methanol | acggcaaagccatgggagcttttcccgaatt |
| | | dehydrogenase | tgaatgccgacatactctcgggatattttccc |
| | | promoter | tgttttttcttagcgcttttcccgtcatct |
| | | | gggtgctgtattccgtaacgtcgcatcccgct |
| | | | ccttccgtatgattaccgtccgtgcgctgccc |
| | | | tctatgaatgattcgttatgcgccttgatca |
| | | | agctaagccggttgtaacaacaaacaccgca |
| | | | atcaatagggggccgcgccgacattatgcgaa |
| | | | aaatcaatctggaggaattATG[. . .]TAA |
| 7 | RBS-P_{mxaF} | M. extorquens | TCTGGAGGAATT |
| | | methanol | |
| | | dehydrogenase | |
| | | ribosome binding site | |
| 8 | RBS-P_{BAD} | ribulokinase promoter | TTTAAGAAGGAGATATACAT |
| | | ribosome binding site | |
| 9 | RBS-ubiC | chorismate pyruvate | GCCGTAGTACCGGCCCAATACAGTACTT |
| | | lyase ribosome | TTTTT |
| | | binding site | |
| 10 | RBS-aroG | DAHP synthase | CACCAAACGAGAAGAACTCAGACTTTTT |
| | | ribosome binding site | T |
| 11 | RBS-aroL | Shikimate kinase 2 | ACCATCTCAAGAGAACTGGCAAGTTCTC |
| | | ribosome binding site | GCACTTTTTTT |
| 12 | RBS-aroD | DHQ dehydratase | AAAACTACGCTCGAGAACGAGTATTATT |
| | | ribosome binding site | TTTTG |
| 13 | Spacers | Spacers | CTCGGATACCCTTACTCTGTTGAAAACG |
| | | | AATAGATAGGTT |
| 14 | Spacers | Spacers | AAGGAACGGTTATTTCTGCGTAGATCTA |
| | | | TCTTACACAGCA |
| 15 | Spacers | Spacers | AGGCAACTGAAACGATTCGGATCCTGTA |
| | | | TTACTATTCTTA |
| 16 | Spacers | Spacers | ACTTTATCTGAGAATAGTCAATCTTCGG |
| | | | AAATCCCAGGTG |
| 17 | Spacers | Spacers | TAAAAGTCTCGTAAAGCGTTCTATCAAT |
| | | | AACCCGTTGGTG |
| 18 | Spacers | Spacers | CCGTCTCAGAATCGGCCGTGAACAATAA |
| | | | AATAGTTTCGGT |
| 19 | Spacers | Spacers | ATTATTGACCACTTCCGAGTAGAATCGT |
| | | | GCTTCAGTAAGA |
| 20 | Tag | Lumio | Gly Ser Gly Ser Cys Cys Pro Gly Cys Cys Gly |
| | | | GlyGSGSCCPGCCGG |
| 21 | Tag | Lumio | GGC TCC GGC TCC TGC TGC CCG GGC |
| | | | TGC TGC GGT GGT |
| 22 | Tag | Lumio | GGG TCG GGG TCG TGT TGT CCC GGG |
| | | | TGT TGT GGC GGC |
| 23 | Tag | Lumio | GGC TCG GGC TCG TGC TGT CCG GGG |
| | | | TGC TGT GGG GGG |
| 24 | Tag | Lumio | GGG TCC GGG TCG TGT TGC CCC GGC |
| | | | TGT TGC |
| 25 | Tag | Lumio-spacer | Gly Ser Gly Ser |
| | | | GSGS |
| 26 | Tag | Lumio-spacer | GGC TCC GGC TCC |
| 27 | Tag | Lumio-spacer | GGG TCG GGG TCG |
| 28 | Tag | Lumio-spacer | GGC TCG GGC TCG |
| 29 | Tag | Lumio-spacer | GGG TCC GGG TCG |
| 30 | Tag | Lumio-Tetra-Cystein | Cys Cys Pro Gly Cys Cys |
| | | | CCPGCC |
| 31 | Tag | Lumio-Tetra-Cystein | TGC TGC CCG GGC TGC TGC |
| 32 | Tag | Lumio-Tetra-Cystein | TGT TGT CCC GGG TGT TGT |
| 33 | Tag | Lumio-Tetra-Cystein | TGC TGT CCG GGG TGC TGT |
| 34 | Tag | Lumio-Tetra-Cystein | TGT TGC CCC GGC TGT TGC |
| 35 | Tag | Lumio-spacer | Gly Gly |
| | | | GG |
| 36 | Tag | Lumio-spacer | GGT GGT |
| 37 | Tag | Lumio-spacer | GGC GGC |
| 38 | Tag | Lumio-spacer | GGG GGG |
| 39 | Tag | 6xHis | CAT CAC CAT CAC CAT CAC |
| 40 | Tag | 6xHis | CAC CAT CAC CAT CAC CAT |
| 41 | Terminators | L3S2P51 | CTCGGTACCAAAAAAAAAAAAAAAGAC |
| | | | GCTGAAAAGCGTCTTTTTTCGTTTTGGTCC |
| 42 | Terminators | rrnB T2/T3Te | AGAAGGCCATCCTGACGGATGGCCTTTg |
| | | | gctcaccttcacgggtgggcctttcttcg |
| 43 | Terminators | rrnB T2 | AGAAGGCCATCCTGACGGATGGCCTTT |
| 44 | Terminators | T3Te | ggctcaccttcacgggtgggcctttcttcg |
| 45 | gBlock | codon-optimised | ggcgccccagctggcaattccaattaaaccg |
| | | genes | ggaatgatgtcggatatttaacggcaaagccat |
| | | | gggagcttttcccgaatttgaatgccgacatact |
| | | | ctcgggatattttccctgttttttcttagcg |
| | | | cttttcccgtcatctgggtgctgtattccgta |
| | | | acgtcgcatcccgctccttccgtatgatt |
| | | | accgtccgtgcgctgccctctatgaatgattcgt |
| | | | tatgcgccttgatcaagctaagccggttgtaac |
| | | | aacaaacaccgcaatcaatagggggccgcgcc |
| | | | gacattatgcgaaaaatcaatctggaggaattGCC |
| | | | GTAGTACCGGCCCAATACAGTACTTTT |
| | | | TTTATGCATGAAACCATCTTCACGCACC |
| | | | ATCCGATTGACTGGTTGAACGAAGACGA |
| | | | CGAGAGCGTCCCCAACTCCGTGCTGGAT |
| | | | TGGCTGCAGGAACGCGGTTCCATGACGA |
| | | | AACGTTTCGAACAGCATTGCCAAAAGGT |
| | | | CACGGTCATCCCGTACCTGGAGCGCTAC |
| | | | ATCACGCCCGAGATGCTCTCGGCGGACG |
| | | | AGGCGGAACGCCTGCCGGAATCCCAAC |
| | | | GCTATTGGCTCCGCGAGGTCATCATGTA |
| | | | TGGCGATAACATCCCGTGGCTGATCGGA |
| | | | CGCACGCTGATCCCGGAAGAGACGCTG |
| | | | ACCAACGATGACAAAAAGCTGGTGGAC |
| | | | ATCGGTCGGGTGCCGTTGGGCCGTTATC |
| | | | TGTTCTCCCACGACTCGTTGACCCGCGA |
| | | | TTACATCGATATCGGCACCAGCGCCGAC |
| | | | TGAGCCAGAAGCCCCTGCTGCTGACGGA |
| | | | AATCTTTCTGCCGGAATCCCCCGCCTAT |
| | | | CGCTAATGCTGCCCGGGCTGCTGCTAAC |
| | | | ACCAAACGAGAAGAACTCAGACTTTT |
| | | | TTATGAACTACCAGAACGATGACCTGCG |
| | | | GATTAAGGAAATCAAGGAGTTGCTGCCG |
| | | | CCCGTCGCCCTGCTGGAGAAATTCCCGG |
| | | | CCACCGAAAACGCGGCCAACACCGTCG |
| | | | CCCATGCCCGCAAAGCGATCCACAAGAT |
| | | | CCTGAAGGGCAACGATGACCGTTTGTTG |
| | | | GTCGTGATCGGCCCCTGCAGCATTCACG |
| | | | ATCCGGTCGCCGCGAAAGAATACGCCAC |
| | | | CCGTTTGCTGGCGTTGCGGGAGGAACTC |
| | | | AAGGATGAGTTGGAAATCGTCATGCGTG |
| | | | TGTACTTTGAAAAACCGCGGACCACCGT |
| | | | GGGCTGGAAGGGTTTGATTAATGACCCG |
| | | | CACATGGATAACAGCTTCCAGATCAACG |
| | | | ACGGTCTGCGTATCGCGCGGAAATTGCT |
| | | | CCTGGACATCAACGACAGCGGATTGCCC |
| | | | GCGGCCGGCGAATTTTTGGACATGATCA |
| | | | CCCCGCAATACCTGGCCGACCTGATGTC |
| | | | GTGGGGTGCCATCGGCGCCCGCACGACC |
| | | | GAATCCCAGGTCCACCGCGAACTCGCCA |
| | | | GCGGTCTGTTCTGTCCGGTCGGTTTCAA |
| | | | AAACGGGACCGACGGGACGATCAAGGT |
| | | | GGCCATCGACGCGATCAATGCGGCCGG |
| | | | AGCCCCCCACTGCTTCCTGAGCGTCACC |
| | | | AAGTGGGGTCATAGCGCCATCGTCAACA |
| | | | CGTCCGGCAACGGCGATTGCCATATCAT |
| | | | CCTGCGGGGCGGTAAGGAGCCCAACTA |
| | | | CAGCGCCAAGCATGTCGCCGAAGTCAA |
| | | | GGAAGGGCTCAACAAGGCCGGACTGCC |
| | | | GGCCCAGGTGATGATCGACTTTAGCCAC |
| | | | GCCAATTCGAGCAAGCAGTTCAAGAAA |
| | | | CAAATGGATGTGTGCGCGGACGTCTGTC |
| | | | AACAGATCGCGGGTGGTGAAAAGGCCA |
| | | | TCATCGGTGTGATGGTCGAAAGCCACCT |
| | | | GGTGGAAGGCAACCAGTCCCTCGAATCC |
| | | | GGCGAGCCCCTGGCCTACGGAAAATCG |
| | | | ATCACCGACGCGTGCATCGGGTGGGAG |
| | | | GATACGGATGCCCTGTTGCGTCAGCTGG |
| | | | CCAATGCGGTCAAGGCCCGGCGCGGTTA |
| | | | ATGTTGTCCCGGGTGTTGTTAAACCATCT |
| | | | CAAGAGAACTGGCAAGTTCTCGCACT |
| | | | TTTTTTATGACCCAGCCCCTGTTTCTGA |
| | | | CGCTGGGTCCGGCGGTCGTTGCTGCGGC |
| | | | TCGGCCCCCGTGGTTGTGGAAAGACGAC |
| | | | GGTCGGGATGGCGCTGGCCGACAGCCTG |
| | | | AATCGCCGTTTCGTCGACACGGATCAGT |
| | | | GGCTGCAGTCGCAGCTGAACATGACGGT |
| | | | GGCGGAAATCGTGGAACGGGAAGAATG |
| | | | GGCCGGCTTTCGCGCCCGGGAGACCGCC |
| | | | GCCCTGGAAGCGGTCACCGCCCCGAGCA |
| | | | CGGTCATTGCCACCGGCGGTGGCATCAT |
| | | | CCTGACCGAATTTAACCGCCATTTCATG |
| | | | CAGAATAATGGTATCGTGGTCTACCTGT |
| | | | GTGCCCCGGTGTCGGTCTTGGTGAATCG |
| | | | CCTCCAGGCGGCCCCCGAGGAAGACTTG |
| | | | CGTCCGACCTTGACGGGCAAACCCCTGT |
| | | | CGGAGGAAGTGCAGGAAGTCCTGGAGG |
| | | | AACGGGATGCCTTGTACCGGGAAGTGGC |
| | | | CCACATCATCATCGACGCCACCAACGAG |
| | | | CCGTCGCAGGTGATCTCGGAAATCCGTA |
| | | | GCGCCCTGGCCCAGACCATCAACTGCTA |
| | | | ATGCTGTCCGGGGTGCTGTTAAAAAACT |
| | | | ACGCTCGAGAACGAGTATTATTTTTTG |
| | | | ATGAAAACCGTCACGGTCAAAGATTTGG |
| | | | TGATTGGTACGGGTGCGCCCAAAATCAT |
| | | | CGTCTCCCTGATGGCGAAAGACATCGCG |
| | | | AGCGTGAAGAGCGAAGCGTTGGCGTAC |
| | | | CGGGAAGCGGACTTCGATATCTTGGAAT |
| | | | GGCGCGTGGACCACTACGCCGACCTGTC |
| | | | GAACGTGGAATCCGTGATGGCCGCCGCG |
| | | | AAGATTTTGCGCGAGACCATGCCGGAGA |
| | | | AGCCCTTGCTGTTTACCTTCCGTTCGGCC |
| | | | AAGGAAGGCGGCGAGCAGGCCATTTCG |
| | | | ACCGAGGCCTATATCGCCCTCAACCGCG |
| | | | CCGCCATCGATTCCGGCCTCGTGGACAT |
| | | | GATCGACTTGGAACTGTTCACGGGCGAT |
| | | | GACCAAGTCAAGGAAACCGTCGCCTAC |
| | | | GCCCACGCCCACGACGTGAAAGTGGTCA |
| | | | TGTCGAACCACGACTTCCATAAGACGCC |
| | | | GGAAGCCGAGGAAATCATCGCGCGCCT |
| | | | GCGTAAGATGCAGTCGTTCGATGCCGAT |
| | | | ATTCCCAAGATTGCCCTGATGCCGCAGT |
| | | | CCACGTCCGACGTCCTGACGCTGCTGGC |
| | | | CGCCACGCTGGAGATGCAGGAACAGTA |
| | | | TGCGGACCGCCCGATCATCACGATGAGC |
| | | | ATGGCCAAGACGGGAGTGATTAGCCGTT |
| | | | TGGCGGGCGAAGTGTTCGGCAGCGCGG |
| | | | CCACGTTTGGGGCGGTGAAGAAAGCCTC |
| | | | CGCGCCGGGCCAGATTAGCGTGAATGAC |
| | | | TTGCGCACCGTCCTGACCATTTTGCACC |
| | | | AGGCGTAATGTTGCCCCGGCTGTTGCTA |
| | | | Aggatctccaggcatcaaa |
| 46 | T_{db1} | Fragment of the | ccaggcatcaaa |
| | | transcription | |
| | | terminator T1 from the | |
| | | E. coli rrnB gene | |
| 47 | pBBR1_{bb} | Targeting arm | ggcgccccagctggcaattcc |
| 48 | Forward | pBBR1_backbone | taaggatctccaggcatcaaa |
| | primer | | |
| 49 | Reverse | pBBR1_backbone | attggaattgccagctgg |
| | primer | | |
| 50 | Forward | pHBA_cassette | ccagctggcaattccaat |
| | primer | | |
| 51 | Reverse | pHBA_cassette | tttgatgcctggagatccttatta |
| | primer | | |
| 52 | ubiC | Providencia rustigianii | ATGCATGAAACCATCTTCACGCACCATC |
| | | chorismate | CGATTGACTGGTTGAACGAAGACGACG |
| | | pyruvate-lyase | AGAGCGTCCCCAACTCCGTGCTGGATTG |
| | | | GCTGCAGGAACGCGGTTCCATGACGAA |
| | | | ACGTTTCGAACAGCATTGCCAAAAGGTC |
| | | | ACGGTCATCCCGTACCTGGAGCGCTACA |
| | | | TCACGCCCGAGATGCTCTCGGCGGACGA |
| | | | GGCGGAACGCCTGCCGGAATCCCAACG |
| | | | CTATTGGCTCCGCGAGGTCATCATGTAT |
| | | | GGCGATAACATCCCGTGGCTGATCGGAC |
| | | | GCACGCTGATCCCGGAAGAGACGCTGA |
| | | | CCAACGATGACAAAAAGCTGGTGGACA |
| | | | TCGGTCGGGTGCCGTTGGGCCGTTATCT |
| | | | GTTCTCCCACGACTCGTTGACCCGCGAT |
| | | | TACATCGATATCGGCACCAGCGCCGACC |
| | | | GCTGGGTCCGGCGGTCGTTGCTGCGGCT |
| | | | GAGCCAGAAGCCCCTGCTGCTGACGGA |
| | | | AATCTTTCTGCCGGAATCCCCCGCCTAT |
| | | | CGCTAA |
| 53 | aroG | Escherichia coli | ATGAACTACCAGAACGATGACCTGCGG |
| | | feedback-inhibition | ATTAAGGAAATCAAGGAGTTGCTGCCGC |
| | | resistant DAHP | CCGTCGCCCTGCTGGAGAAATTCCCGGC |
| | | synthase | CACCGAAAACGCGGCCAACACCGTCGC |
| | | | CCATGCCCGCAAAGCGATCCACAAGATC |
| | | | CTGAAGGGCAACGATGACCGTTTGTTGG |
| | | | TCGTGATCGGCCCCTGCAGCATTCACGA |
| | | | TCCGGTCGCCGCGAAAGAATACGCCACC |
| | | | CGTTTGCTGGCGTTGCGGGAGGAACTCA |
| | | | AGGATGAGTTGGAAATCGTCATGCGTGT |
| | | | GTACTTTGAAAAACCGCGGACCACCGTG |
| | | | GGCTGGAAGGGTTTGATTAATGACCCGC |
| | | | ACATGGATAACAGCTTCCAGATCAACGA |
| | | | CGGTCTGCGTATCGCGCGGAAATTGCTC |
| | | | CTGGACATCAACGACAGCGGATTGCCCG |
| | | | CGGCCGGCGAATTTTTGGACATGATCAC |
| | | | CCCGCAATACCTGGCCGACCTGATGTCG |
| | | | TGGGGTGCCATCGGCGCCCGCACGACCG |
| | | | AATCCCAGGTCCACCGCGAACTCGCCAG |
| | | | CGGTCTGTTCTGTCCGGTCGGTTTCAAA |
| | | | AACGGGACCGACGGGACGATCAAGGTG |
| | | | GCCATCGACGCGATCAATGCGGCCGGA |
| | | | GCCCCCCACTGCTTCCTGAGCGTCACCA |
| | | | AGTGGGGTCATAGCGCCATCGTCAACAC |
| | | | GTCCGGCAACGGCGATTGCCATATCATC |
| | | | CTGCGGGGCGGTAAGGAGCCCAACTAC |
| | | | AGCGCCAAGCATGTCGCCGAAGTCAAG |
| | | | GAAGGGCTCAACAAGGCCGGACTGCCG |
| | | | GCCCAGGTGATGATCGACTTTAGCCACG |
| | | | CCAATTCGAGCAAGCAGTTCAAGAAAC |
| | | | AAATGGATGTGTGCGCGGACGTCTGTCA |
| | | | ACAGATCGCGGGTGGTGAAAAGGCCAT |
| | | | CATCGGTGTGATGGTCGAAAGCCACCTG |
| | | | GTGGAAGGCAACCAGTCCCTCGAATCCG |
| | | | GCGAGCCCCTGGCCTACGGAAAATCGAT |
| | | | CACCGACGCGTGCATCGGGTGGGAGGA |
| | | | TACGGATGCCCTGTTGCGTCAGCTGGCC |
| | | | AATGCGGTCAAGGCCCGGCGCGGTTAA |
| 54 | aroL | Escherichia coli | ATGACCCAGCCCCTGTTTCTGATCGGCC |
| | | shikimate kinase 2 | CCCGTGGTTGTGGAAAGACGACGGTCGG |
| | | | GATGGCGCTGGCCGACAGCCTGAATCGC |
| | | | CGTTTCGTCGACACGGATCAGTGGCTGC |
| | | | AGTCGCAGCTGAACATGACGGTGGCGG |
| | | | AAATCGTGGAACGGGAAGAATGGGCCG |
| | | | GCTTTCGCGCCCGGGAGACCGCCGCCCT |
| | | | GGAAGCGGTCACCGCCCCGAGCACGGT |
| | | | CATTGCCACCGGCGGTGGCATCATCCTG |
| | | | ACCGAATTTAACCGCCATTTCATGCAGA |
| | | | ATAATGGTATCGTGGTCTACCTGTGTGC |
| | | | CCCGGTGTCGGTCTTGGTGAATCGCCTC |
| | | | CAGGCGGCCCCCGAGGAAGACTTGCGTC |
| | | | CGACCTTGACGGGCAAACCCCTGTCGGA |
| | | | GGAAGTGCAGGAAGTCCTGGAGGAACG |
| | | | GGATGCCTTGTACCGGGAAGTGGCCCAC |
| | | | ATCATCATCGACGCCACCAACGAGCCGT |
| | | | CGCAGGTGATCTCGGAAATCCGTAGCGC |
| | | | CCTGGCCCAGACCATCAACTGCTAA |
| 55 | aroD | Escherichia coli | ATGAAAACCGTCACGGTCAAAGATTTGG |
| | | 3-dehydroquinate | TGATTGGTACGGGTGCGCCCAAAATCAT |
| | | dehydratase | CGTCTCCCTGATGGCGAAAGACATCGCG |
| | | | AGCGTGAAGAGCGAAGCGTTGGCGTAC |
| | | | CGGGAAGCGGACTTCGATATCTTGGAAT |
| | | | GGCGCGTGGACCACTACGCCGACCTGTC |
| | | | GAACGTGGAATCCGTGATGGCCGCCGCG |
| | | | AAGATTTTGCGCGAGACCATGCCGGAGA |
| | | | AGCCCTTGCTGTTTACCTTCCGTTCGGCC |
| | | | AAGGAAGGCGGCGAGCAGGCCATTTCG |
| | | | ACCGAGGCCTATATCGCCCTCAACCGCG |
| | | | CCGCCATCGATTCCGGCCTCGTGGACAT |
| | | | GATCGACTTGGAACTGTTCACGGGCGAT |
| | | | GACCAAGTCAAGGAAACCGTCGCCTAC |
| | | | GCCCACGCCCACGACGTGAAAGTGGTCA |
| | | | TGTCGAACCACGACTTCCATAAGACGCC |
| | | | GGAAGCCGAGGAAATCATCGCGCGCCT |
| | | | GCGTAAGATGCAGTCGTTCGATGCCGAT |
| | | | ATTCCCAAGATTGCCCTGATGCCGCAGT |
| | | | CCACGTCCGACGTCCTGACGCTGCTGGC |
| | | | CGCCACGCTGGAGATGCAGGAACAGTA |
| | | | TGCGGACCGCCCGATCATCACGATGAGC |
| | | | ATGGCCAAGACGGGAGTGATTAGCCGTT |
| | | | TGGCGGGCGAAGTGTTCGGCAGCGCGG |
| | | | CCACGTTTGGGGCGGTGAAGAAAGCCTC |
| | | | CGCGCCGGGCCAGATTAGCGTGAATGAC |
| | | | TTGCGCACCGTCCTGACCATTTTGCACC |
| | | | AGGCGTAA |
| 56 | RBS-ubiC | Ribosome Binding | GCCGTAGTACCGGCCCAATACAGTAC |
| | | Site-Providencia | TTTTTTTATGCATGAAACCATCTTCACG |
| | | rustigianii chorismate | CACCATCCGATTGACTGGTTGAACGAAG |
| | | pyruvate-lyase | ACGACGAGAGCGTCCCCAACTCCGTGCT |
| | | | GGATTGGCTGCAGGAACGCGGTTCCATG |
| | | | ACGAAACGTTTCGAACAGCATTGCCAAA |
| | | | AGGTCACGGTCATCCCGTACCTGGAGCG |
| | | | CTACATCACGCCCGAGATGCTCTCGGCG |
| | | | GACGAGGCGGAACGCCTGCCGGAATCC |
| | | | CAACGCTATTGGCTCCGCGAGGTCATCA |
| | | | TGTATGGCGATAACATCCCGTGGCTGAT |
| | | | CGGACGCACGCTGATCCCGGAAGAGAC |
| | | | GCTGACCAACGATGACAAAAAGCTGGT |
| | | | GGACATCGGTCGGGTGCCGTTGGGCCGT |
| | | | TATCTGTTCTCCCACGACTCGTTGACCCG |
| | | | CGATTACATCGATATCGGCACCAGCGCC |
| | | | GACCGCTGGGTCCGGCGGTCGTTGCTGC |
| | | | GGCTGAGCCAGAAGCCCCTGCTGCTGAC |
| | | | GGAAATCTTTCTGCCGGAATCCCCCGCC |
| | | | TATCGCTAA |
| 57 | RBS-aroG | Ribosome Binding | CACCAAACGAGAAGAACTCAGACTTT |
| | | Site-Escherichia coli | TTTATGAACTACCAGAACGATGACCTGC |
| | | feedback-inhibition | GGATTAAGGAAATCAAGGAGTTGCTGCC |
| | | resistant DAHP | GCCCGTCGCCCTGCTGGAGAAATTCCCG |
| | | synthase | GCCACCGAAAACGCGGCCAACACCGTC |
| | | | GCCCATGCCCGCAAAGCGATCCACAAG |
| | | | ATCCTGAAGGGCAACGATGACCGTTTGT |
| | | | TGGTCGTGATCGGCCCCTGCAGCATTCA |
| | | | CGATCCGGTCGCCGCGAAAGAATACGCC |
| | | | ACCCGTTTGCTGGCGTTGCGGGAGGAAC |
| | | | TCAAGGATGAGTTGGAAATCGTCATGCG |
| | | | TGTGTACTTTGAAAAACCGCGGACCACC |
| | | | GTGGGCTGGAAGGGTTTGATTAATGACC |
| | | | CGCACATGGATAACAGCTTCCAGATCAA |
| | | | CGACGGTCTGCGTATCGCGCGGAAATTG |
| | | | CTCCTGGACATCAACGACAGCGGATTGC |
| | | | CCGCGGCCGGCGAATTTTTGGACATGAT |
| | | | CACCCCGCAATACCTGGCCGACCTGATG |
| | | | TCGTGGGGTGCCATCGGCGCCCGCACGA |
| | | | CCGAATCCCAGGTCCACCGCGAACTCGC |
| | | | CAGCGGTCTGTTCTGTCCGGTCGGTTTC |
| | | | AAAAACGGGACCGACGGGACGATCAAG |
| | | | GTGGCCATCGACGCGATCAATGCGGCCG |
| | | | GAGCCCCCCACTGCTTCCTGAGCGTCAC |
| | | | CAAGTGGGGTCATAGCGCCATCGTCAAC |
| | | | ACGTCCGGCAACGGCGATTGCCATATCA |
| | | | TCCTGCGGGGCGGTAAGGAGCCCAACTA |
| | | | CAGCGCCAAGCATGTCGCCGAAGTCAA |
| | | | GGAAGGGCTCAACAAGGCCGGACTGCC |
| | | | GGCCCAGGTGATGATCGACTTTAGCCAC |
| | | | GCCAATTCGAGCAAGCAGTTCAAGAAA |
| | | | CAAATGGATGTGTGCGCGGACGTCTGTC |
| | | | AACAGATCGCGGGTGGTGAAAAGGCCA |
| | | | TCATCGGTGTGATGGTCGAAAGCCACCT |
| | | | GGTGGAAGGCAACCAGTCCCTCGAATCC |
| | | | GGCGAGCCCCTGGCCTACGGAAAATCG |
| | | | ATCACCGACGCGTGCATCGGGTGGGAG |
| | | | GATACGGATGCCCTGTTGCGTCAGCTGG |
| | | | CCAATGCGGTCAAGGCCCGGCGCGGTTAA |
| 58 | RBS-aroL | Ribosome Binding | ACCATCTCAAGAGAACTGGCAAGTTC |
| | | shikimate kinase 2 | TCGCACTTTTTTTATGACCCAGCCCCTG |
| | | Site-Escherichia coli | TTTCTGATCGGCCCCCGTGGTTGTGGAA |
| | | | AGACGACGGTCGGGATGGCGCTGGCCG |
| | | | ACAGCCTGAATCGCCGTTTCGTCGACAC |
| | | | GGATCAGTGGCTGCAGTCGCAGCTGAAC |
| | | | ATGACGGTGGCGGAAATCGTGGAACGG |
| | | | GAAGAATGGGCCGGCTTTCGCGCCCGGG |
| | | | AGACCGCCGCCCTGGAAGCGGTCACCGC |
| | | | CCCGAGCACGGTCATTGCCACCGGCGGT |
| | | | GGCATCATCCTGACCGAATTTAACCGCC |
| | | | ATTTCATGCAGAATAATGGTATCGTGGT |
| | | | CTACCTGTGTGCCCCGGTGTCGGTCTTG |
| | | | GTGAATCGCCTCCAGGCGGCCCCCGAGG |
| | | | AAGACTTGCGTCCGACCTTGACGGGCAA |
| | | | ACCCCTGTCGGAGGAAGTGCAGGAAGT |
| | | | CCTGGAGGAACGGGATGCCTTGTACCGG |
| | | | GAAGTGGCCCACATCATCATCGACGCCA |
| | | | CCAACGAGCCGTCGCAGGTGATCTCGGA |
| | | | AATCCGTAGCGCCCTGGCCCAGACCATC |
| | | | AACTGCTAA |
| 59 | RBS-aroD | Ribosome Binding | AAAACTACGCTCGAGAACGAGTATTA |
| | | Site-Escherichia coli | TTTTTTGATGAAAACCGTCACGGTCAAA |
| | | 3-dehydroquinate | GATTTGGTGATTGGTACGGGTGCGCCCA |
| | | dehydratase | AAATCATCGTCTCCCTGATGGCGAAAGA |
| | | | CATCGCGAGCGTGAAGAGCGAAGCGTT |
| | | | GGCGTACCGGGAAGCGGACTTCGATATC |
| | | | TTGGAATGGCGCGTGGACCACTACGCCG |
| | | | ACCTGTCGAACGTGGAATCCGTGATGGC |
| | | | CGCCGCGAAGATTTTGCGCGAGACCATG |
| | | | CCGGAGAAGCCCTTGCTGTTTACCTTCC |
| | | | GTTCGGCCAAGGAAGGCGGCGAGCAGG |
| | | | CCATTTCGACCGAGGCCTATATCGCCCT |
| | | | CAACCGCGCCGCCATCGATTCCGGCCTC |
| | | | GTGGACATGATCGACTTGGAACTGTTCA |
| | | | CGGGCGATGACCAAGTCAAGGAAACCG |
| | | | TCGCCTACGCCCACGCCCACGACGTGAA |
| | | | AGTGGTCATGTCGAACCACGACTTCCAT |
| | | | AAGACGCCGGAAGCCGAGGAAATCATC |
| | | | GCGCGCCTGCGTAAGATGCAGTCGTTCG |
| | | | ATGCCGATATTCCCAAGATTGCCCTGAT |
| | | | GCCGCAGTCCACGTCCGACGTCCTGACG |
| | | | CTGCTGGCCGCCACGCTGGAGATGCAGG |
| | | | AACAGTATGCGGACCGCCCGATCATCAC |
| | | | GATGAGCATGGCCAAGACGGGAGTGAT |
| | | | TAGCCGTTTGGCGGGCGAAGTGTTCGGC |
| | | | AGCGCGGCCACGTTTGGGGCGGTGAAG |
| | | | AAAGCCTCCGCGCCGGGCCAGATTAGCG |
| | | | TGAATGACTTGCGCACCGTCCTGACCAT |
| | | | TTTGCACCAGGCGTAA |
| 60 | ubiC-Tag | Tagged-Providencia | ATGCATGAAACCATCTTCACGCACCATC |
| | | rustigianii chorismate | CGATTGACTGGTTGAACGAAGACGACG |
| | | pyruvate-lyase | AGAGCGTCCCCAACTCCGTGCTGGATTG |
| | | | GCTGCAGGAACGCGGTTCCATGACGAA |
| | | | ACGTTTCGAACAGCATTGCCAAAAGGTC |
| | | | ACGGTCATCCCGTACCTGGAGCGCTACA |
| | | | TCACGCCCGAGATGCTCTCGGCGGACGA |
| | | | GGCGGAACGCCTGCCGGAATCCCAACG |
| | | | CTATTGGCTCCGCGAGGTCATCATGTAT |
| | | | GGCGATAACATCCCGTGGCTGATCGGAC |
| | | | GCACGCTGATCCCGGAAGAGACGCTGA |
| | | | CCAACGATGACAAAAAGCTGGTGGACA |
| | | | TCGGTCGGGTGCCGTTGGGCCGTTATCT |
| | | | GTTCTCCCACGACTCGTTGACCCGCGAT |
| | | | TACATCGATATCGGCACCAGCGCCGACC |
| | | | GCTGGGTCCGGCGGTCGTTGCTGCGGCT |
| | | | GAGCCAGAAGCCCCTGCTGCTGACGGA |
| | | | AATCTTTCTGCCGGAATCCCCCGCCTAT |
| | | | CGCTAATGCTGCCCGGGCTGCTGCTAA |
| 61 | aroG-Tag | Tagged-Escherichia | ATGAACTACCAGAACGATGACCTGCGG |
| | | coli feedback- | ATTAAGGAAATCAAGGAGTTGCTGCCGC |
| | | inhibition resistant | CCGTCGCCCTGCTGGAGAAATTCCCGGC |
| | | DAHP synthase | CACCGAAAACGCGGCCAACACCGTCGC |
| | | | CCATGCCCGCAAAGCGATCCACAAGATC |
| | | | CTGAAGGGCAACGATGACCGTTTGTTGG |
| | | | TCGTGATCGGCCCCTGCAGCATTCACGA |
| | | | TCCGGTCGCCGCGAAAGAATACGCCACC |
| | | | CGTTTGCTGGCGTTGCGGGAGGAACTCA |
| | | | AGGATGAGTTGGAAATCGTCATGCGTGT |
| | | | GTACTTTGAAAAACCGCGGACCACCGTG |
| | | | GGCTGGAAGGGTTTGATTAATGACCCGC |
| | | | ACATGGATAACAGCTTCCAGATCAACGA |
| | | | CGGTCTGCGTATCGCGCGGAAATTGCTC |
| | | | CTGGACATCAACGACAGCGGATTGCCCG |
| | | | CGGCCGGCGAATTTTTGGACATGATCAC |
| | | | CCCGCAATACCTGGCCGACCTGATGTCG |
| | | | TGGGGTGCCATCGGCGCCCGCACGACCG |
| | | | AATCCCAGGTCCACCGCGAACTCGCCAG |
| | | | CGGTCTGTTCTGTCCGGTCGGTTTCAAA |
| | | | AACGGGACCGACGGGACGATCAAGGTG |
| | | | GCCATCGACGCGATCAATGCGGCCGGA |
| | | | GCCCCCCACTGCTTCCTGAGCGTCACCA |
| | | | AGTGGGGTCATAGCGCCATCGTCAACAC |
| | | | GTCCGGCAACGGCGATTGCCATATCATC |
| | | | CTGCGGGGCGGTAAGGAGCCCAACTAC |
| | | | AGCGCCAAGCATGTCGCCGAAGTCAAG |
| | | | GAAGGGCTCAACAAGGCCGGACTGCCG |
| | | | GCCCAGGTGATGATCGACTTTAGCCACG |
| | | | CCAATTCGAGCAAGCAGTTCAAGAAAC |
| | | | AAATGGATGTGTGCGCGGACGTCTGTCA |
| | | | ACAGATCGCGGGTGGTGAAAAGGCCAT |
| | | | CATCGGTGTGATGGTCGAAAGCCACCTG |
| | | | GTGGAAGGCAACCAGTCCCTCGAATCCG |
| | | | GCGAGCCCCTGGCCTACGGAAAATCGAT |
| | | | CACCGACGCGTGCATCGGGTGGGAGGA |
| | | | TACGGATGCCCTGTTGCGTCAGCTGGCC |
| | | | AATGCGGTCAAGGCCCGGCGCGGTTAAT |
| | | | GTTGTCCCGGGTGTTGTTAA |
| 62 | aroL-Tag | Tagged-Escherichia | ATGACCCAGCCCCTGTTTCTGATCGGCC |
| | | coli shikimate kinase 2 | CCCGTGGTTGTGGAAAGACGACGGTCGG |
| | | | GATGGCGCTGGCCGACAGCCTGAATCGC |
| | | | CGTTTCGTCGACACGGATCAGTGGCTGC |
| | | | AGTCGCAGCTGAACATGACGGTGGCGG |
| | | | AAATCGTGGAACGGGAAGAATGGGCCG |
| | | | GCTTTCGCGCCCGGGAGACCGCCGCCCT |
| | | | GGAAGCGGTCACCGCCCCGAGCACGGT |
| | | | CATTGCCACCGGCGGTGGCATCATCCTG |
| | | | ACCGAATTTAACCGCCATTTCATGCAGA |
| | | | ATAATGGTATCGTGGTCTACCTGTGTGC |
| | | | CCCGGTGTCGGTCTTGGTGAATCGCCTC |
| | | | CAGGCGGCCCCCGAGGAAGACTTGCGTC |
| | | | CGACCTTGACGGGCAAACCCCTGTCGGA |
| | | | GGAAGTGCAGGAAGTCCTGGAGGAACG |
| | | | GGATGCCTTGTACCGGGAAGTGGCCCAC |
| | | | ATCATCATCGACGCCACCAACGAGCCGT |
| | | | CGCAGGTGATCTCGGAAATCCGTAGCGC |
| | | | CCTGGCCCAGACCATCAACTGCTAATGC |
| | | | TGTCCGGGGTGCTGTTAA |
| 63 | aroD-Tag | Tagged-Escherichia | ATGAAAACCGTCACGGTCAAAGATTTGG |
| | | coli 3-dehydroquinate | TGATTGGTACGGGTGCGCCCAAAATCAT |
| | | dehydratase | CGTCTCCCTGATGGCGAAAGACATCGCG |
| | | | AGCGTGAAGAGCGAAGCGTTGGCGTAC |
| | | | CGGGAAGCGGACTTCGATATCTTGGAAT |
| | | | GGCGCGTGGACCACTACGCCGACCTGTC |
| | | | GAACGTGGAATCCGTGATGGCCGCCGCG |
| | | | AAGATTTTGCGCGAGACCATGCCGGAGA |
| | | | AGCCCTTGCTGTTTACCTTCCGTTCGGCC |
| | | | AAGGAAGGCGGCGAGCAGGCCATTTCG |
| | | | ACCGAGGCCTATATCGCCCTCAACCGCG |
| | | | CCGCCATCGATTCCGGCCTCGTGGACAT |
| | | | GATCGACTTGGAACTGTTCACGGGCGAT |
| | | | GACCAAGTCAAGGAAACCGTCGCCTAC |
| | | | GCCCACGCCCACGACGTGAAAGTGGTCA |
| | | | TGTCGAACCACGACTTCCATAAGACGCC |
| | | | GGAAGCCGAGGAAATCATCGCGCGCCT |
| | | | GCGTAAGATGCAGTCGTTCGATGCCGAT |
| | | | ATTCCCAAGATTGCCCTGATGCCGCAGT |
| | | | CCACGTCCGACGTCCTGACGCTGCTGGC |
| | | | CGCCACGCTGGAGATGCAGGAACAGTA |
| | | | TGCGGACCGCCCGATCATCACGATGAGC |
| | | | ATGGCCAAGACGGGAGTGATTAGCCGTT |
| | | | TGGCGGGCGAAGTGTTCGGCAGCGCGG |
| | | | CCACGTTTGGGGCGGTGAAGAAAGCCTC |
| | | | CGCGCCGGGCCAGATTAGCGTGAATGAC |
| | | | TTGCGCACCGTCCTGACCATTTTGCACC |
| | | | AGGCGTAATGTTGCCCCGGCTGTTGCTAA |
| 64 | RBS-ubiC- | Providencia | GCCGTAGTACCGGCCCAATACAGTAC |
| | Tag | rustigianii chorismate | TTTTTTTATGCATGAAACCATCTTCACG |
| | | pyruvate-lyase | CACCATCCGATTGACTGGTTGAACGAAG |
| | | | ACGACGAGAGCGTCCCCAACTCCGTGCT |
| | | | GGATTGGCTGCAGGAACGCGGTTCCATG |
| | | | ACGAAACGTTTCGAACAGCATTGCCAAA |
| | | | AGGTCACGGTCATCCCGTACCTGGAGCG |
| | | | CTACATCACGCCCGAGATGCTCTCGGCG |
| | | | GACGAGGCGGAACGCCTGCCGGAATCC |
| | | | CAACGCTATTGGCTCCGCGAGGTCATCA |
| | | | TGTATGGCGATAACATCCCGTGGCTGAT |
| | | | CGGACGCACGCTGATCCCGGAAGAGAC |
| | | | GCTGACCAACGATGACAAAAAGCTGGT |
| | | | GGACATCGGTCGGGTGCCGTTGGGCCGT |
| | | | TATCTGTTCTCCCACGACTCGTTGACCCG |
| | | | CGATTACATCGATATCGGCACCAGCGCC |
| | | | GACCGCTGGGTCCGGCGGTCGTTGCTGC |
| | | | GGCTGAGCCAGAAGCCCCTGCTGCTGAC |
| | | | GGAAATCTTTCTGCCGGAATCCCCCGCC |
| | | | TATCGCTAATGCTGCCCGGGCTGCTGCTAA |
| 65 | RBS-aroG- | Escherichia coli | CACCAAACGAGAAGAACTCAGACTTT |
| | Tag | feedback-inhibition | TTTATGAACTACCAGAACGATGACCTGC |
| | | resistant DAHP | GGATTAAGGAAATCAAGGAGTTGCTGCC |
| | | synthase | GCCCGTCGCCCTGCTGGAGAAATTCCCG |
| | | | GCCACCGAAAACGCGGCCAACACCGTC |
| | | | GCCCATGCCCGCAAAGCGATCCACAAG |
| | | | ATCCTGAAGGGCAACGATGACCGTTTGT |
| | | | TGGTCGTGATCGGCCCCTGCAGCATTCA |
| | | | CGATCCGGTCGCCGCGAAAGAATACGCC |
| | | | ACCCGTTTGCTGGCGTTGCGGGAGGAAC |
| | | | TCAAGGATGAGTTGGAAATCGTCATGCG |
| | | | TGTGTACTTTGAAAAACCGCGGACCACC |
| | | | GTGGGCTGGAAGGGTTTGATTAATGACC |
| | | | CGCACATGGATAACAGCTTCCAGATCAA |
| | | | CGACGGTCTGCGTATCGCGCGGAAATTG |
| | | | CTCCTGGACATCAACGACAGCGGATTGC |
| | | | CCGCGGCCGGCGAATTTTTGGACATGAT |
| | | | CACCCCGCAATACCTGGCCGACCTGATG |
| | | | TCGTGGGGTGCCATCGGCGCCCGCACGA |
| | | | CCGAATCCCAGGTCCACCGCGAACTCGC |
| | | | CAGCGGTCTGTTCTGTCCGGTCGGTTTC |
| | | | AAAAACGGGACCGACGGGACGATCAAG |
| | | | GTGGCCATCGACGCGATCAATGCGGCCG |
| | | | GAGCCCCCCACTGCTTCCTGAGCGTCAC |
| | | | CAAGTGGGGTCATAGCGCCATCGTCAAC |
| | | | ACGTCCGGCAACGGCGATTGCCATATCA |
| | | | TCCTGCGGGGCGGTAAGGAGCCCAACTA |
| | | | CAGCGCCAAGCATGTCGCCGAAGTCAA |
| | | | GGAAGGGCTCAACAAGGCCGGACTGCC |
| | | | GGCCCAGGTGATGATCGACTTTAGCCAC |
| | | | GCCAATTCGAGCAAGCAGTTCAAGAAA |
| | | | CAAATGGATGTGTGCGCGGACGTCTGTC |
| | | | AACAGATCGCGGGTGGTGAAAAGGCCA |
| | | | TCATCGGTGTGATGGTCGAAAGCCACCT |
| | | | GGTGGAAGGCAACCAGTCCCTCGAATCC |
| | | | GGCGAGCCCCTGGCCTACGGAAAATCG |
| | | | ATCACCGACGCGTGCATCGGGTGGGAG |
| | | | GATACGGATGCCCTGTTGCGTCAGCTGG |
| | | | CCAATGCGGTCAAGGCCCGGCGCGGTTA |
| | | | ATGTTGTCCCGGGTGTTGTTAA |
| 66 | RBS-aroL- | Escherichia coli | ACCATCTCAAGAGAACTGGCAAGTTC |
| | Tag | shikimate kinase 2 | TCGCACTTTTTTTATGACCCAGCCCCTG |
| | | | TTTCTGATCGGCCCCCGTGGTTGTGGAA |
| | | | AGACGACGGTCGGGATGGCGCTGGCCG |
| | | | ACAGCCTGAATCGCCGTTTCGTCGACAC |
| | | | GGATCAGTGGCTGCAGTCGCAGCTGAAC |
| | | | ATGACGGTGGCGGAAATCGTGGAACGG |
| | | | GAAGAATGGGCCGGCTTTCGCGCCCGGG |
| | | | AGACCGCCGCCCTGGAAGCGGTCACCGC |
| | | | CCCGAGCACGGTCATTGCCACCGGCGGT |
| | | | GGCATCATCCTGACCGAATTTAACCGCC |
| | | | ATTTCATGCAGAATAATGGTATCGTGGT |
| | | | CTACCTGTGTGCCCCGGTGTCGGTCTTG |
| | | | GTGAATCGCCTCCAGGCGGCCCCCGAGG |
| | | | AAGACTTGCGTCCGACCTTGACGGGCAA |
| | | | ACCCCTGTCGGAGGAAGTGCAGGAAGT |
| | | | CCTGGAGGAACGGGATGCCTTGTACCGG |
| | | | GAAGTGGCCCACATCATCATCGACGCCA |
| | | | CCAACGAGCCGTCGCAGGTGATCTCGGA |
| | | | AATCCGTAGCGCCCTGGCCCAGACCATC |
| | | | AACTGCTAATGCTGTCCGGGGTGCTGTTAA |
| 67 | RBS-aroD- | Escherichia coli 3- | AAAACTACGCTCGAGAACGAGTATTA |
| | Tag | dehydroquinate | TTTTTTGATGAAAACCGTCACGGTCAAA |
| | | dehydratase | GATTTGGTGATTGGTACGGGTGCGCCCA |
| | | | AAATCATCGTCTCCCTGATGGCGAAAGA |
| | | | CATCGCGAGCGTGAAGAGCGAAGCGTT |
| | | | GGCGTACCGGGAAGCGGACTTCGATATC |
| | | | TTGGAATGGCGCGTGGACCACTACGCCG |
| | | | ACCTGTCGAACGTGGAATCCGTGATGGC |
| | | | CGCCGCGAAGATTTTGCGCGAGACCATG |
| | | | CCGGAGAAGCCCTTGCTGTTTACCTTCC |
| | | | GTTCGGCCAAGGAAGGCGGCGAGCAGG |
| | | | CCATTTCGACCGAGGCCTATATCGCCCT |
| | | | CAACCGCGCCGCCATCGATTCCGGCCTC |
| | | | GTGGACATGATCGACTTGGAACTGTTCA |
| | | | CGGGCGATGACCAAGTCAAGGAAACCG |
| | | | TCGCCTACGCCCACGCCCACGACGTGAA |
| | | | AGTGGTCATGTCGAACCACGACTTCCAT |
| | | | AAGACGCCGGAAGCCGAGGAAATCATC |
| | | | GCGCGCCTGCGTAAGATGCAGTCGTTCG |
| | | | ATGCCGATATTCCCAAGATTGCCCTGAT |
| | | | GCCGCAGTCCACGTCCGACGTCCTGACG |
| | | | CTGCTGGCCGCCACGCTGGAGATGCAGG |
| | | | AACAGTATGCGGACCGCCCGATCATCAC |
| | | | GATGAGCATGGCCAAGACGGGAGTGAT |
| | | | TAGCCGTTTGGCGGGCGAAGTGTTCGGC |
| | | | AGCGCGGCCACGTTTGGGGCGGTGAAG |
| | | | AAAGCCTCCGCGCCGGGCCAGATTAGCG |
| | | | TGAATGACTTGCGCACCGTCCTGACCAT |
| | | | TTTGCACCAGGCGTAATGTTGCCCCGGC |
| | | | TGTTGCTAA |
| 70 | Kan_fwd | pCM66T vector | gcaccatgttggaatttaatcgc |
| 71 | Kan_rev | pCM66T vector | gcgattaaartccaacatggatgc |
| 72 | PmxcaF for | pCM66T vector | aattaaaccgggaatgatgt |
| 73 | dbl-term rev | pCM66T vector | cgttttatttgatgcctgga |
| 74 | dbl-term rev | pCM66T vector | ctccaggcatcaaataaaacgaaagg |
| 75 | PmxcaF- | pCM66T vector | cattcccggtttaattattgcgttgcgctcac |
| | pCM66T rev | | |
| 76 | ubiC_seq for | pCM66T vector | CTCTCGGCCGACGAAGCCGAAC |
| 77 | ubiC_seq rev | pCM66T vector | CCGATCAGCCAGGGGATGTTATCG |

While certain embodiments have been illustrated and described, it should be understood that changes and modifications can be made therein in accordance with ordinary skill in the art without departing from the technology in its broader aspects.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A method of producing para-hydroxybenzoic acid (pHBA) or a derivative thereof, the method comprising:
culturing a recombinant microorganism in a fermentation broth;
adding a carbon source to the fermentation broth; and
isolating the pHBA from the fermentation broth;
wherein the recombinant microorganism comprises a genetically engineered pathway comprising a nucleic acid sequence encoding an exogenous 3-deoxy-D-arabino-heptulosonate-7-phosphate (DAHP) synthase of EC 4.1.2.15, or EC 2.5.1.54; and
wherein the exogenous DAHP synthase of EC 4.1.2.15, or EC 2.5.1.54 comprises a feedback-inhibition resistant mutation in the wild-type amino acid sequence of the DAHP synthase.

2. (canceled)

3. The method of claim 1, wherein
the feedback-inhibition resistant mutation is a substitution at position 180 of the wild-type amino acid sequence of DAHP synthase;
the feedback-inhibition resistant mutation is a serine to phenylalanine mutation at position 180 of the wild-type amino acid sequence of DAHP synthase; or
the wild-type amino acid sequence of DAHP synthase comprises the amino acid sequence set forth in SEQ ID NO: 1.

4. (canceled)

5. The method of claim 48, wherein
the chorismate pyruvate lyase is P. rustigianii UbiC, C. sakazakii UbiC, or comprises the amino acid sequence of SEQ ID NO: 4 or 5;
the DAHP synthase is E. coli AroG, or comprises the amino acid sequence of SEQ ID NO: 1;
the shikimate kinase is E. coli AroL, or comprises the amino acid sequence of SEQ ID NO: 3; or
the DHQ is E. coli AroD, or comprises the amino acid sequence of SEQ ID NO: 2.

6. (canceled)

7. The method of claim 1, wherein the genetically engineered pathway:
is encoded by a single vector driven by a single promoter, wherein the promoter is:
a constitutive promoter;
an inducible promoter;
selected from a M. extorquens methanol dehydrogenase promoter (PmxaF), an E. coli (P_{BAD}) ribulokinase promoter (araBp), an E. coli (P_{lac}) β-galactosidase promoter (lacZp), or a bacteriophage lambda promoter (λP_{L}); or
encoded by a nucleic acid sequence set forth in SEQ ID NO: 6; or
comprises a nucleic acid sequence selected from SEQ ID NOs: 6, 45, 52, 53, 54, 55, 56, 57, 58, 59, 60, 61, 62, 63, 64, 65, 66, 67, or a combination of any two or more thereof.

8. The method of claim 7, wherein the single vector comprises at least two, at least three, at least four, or at least five nucleic sequences each encoding a polypeptide.

9.-13. (canceled)

14. The method of claim 1, wherein the recombinant microorganism is Methylomicrobium alcahphilum 20Z.

15. The method of claim 1, wherein the recombinant microorganism comprises a nucleic acid sequence set forth in SEQ ID NO: 6 and a nucleic acid sequence encoding the polypeptide of:
SEQ ID NO:4;
SEQ ID NO: 5;
SEQ ID NOs: 1 and 4;
SEQ ID NOs: 1 and 5;
SEQ ID NOs: 2 and 4;
SEQ ID NOs: 2 and 5;
SEQ ID NOs: 3 and 4;
SEQ ID NOs: 3 and 5;
SEQ ID NOs: 1, 3, and 4;
SEQ ID NOs: 1, 3, and 5;
SEQ ID NOs: 1, 2, and 4
SEQ ID NOs: 1, 2, and 5;
SEQ ID NOs: 2, 3, and 4;
SEQ ID NOs: 2, 3, and 5;
SEQ ID NOs: 1, 2, 3, and 4; or
SEQ ID NOs: 1, 2, 3, and 5.

16. The method of claim 1, wherein the recombinant microorganism comprises SEQ ID NO: 45.

17.-21. (canceled)

22. The method of claim 1, wherein the recombinant microorganism comprises an exogenous amino acid sequence comprising SEQ ID NOs: 1, 2, 3, 4, 5, or a combination of any two or more thereof.

23. A recombinant microorganism for producing para-hydroxybenzoic acid (pHBA) or a derivative thereof, the recombinant microorganism comprising a genetically engineered pathway comprising a nucleic acid sequence encoding an exogenous 3-deoxy-D-arabino-heptulosonate-7-phosphate (DAHP) synthase of EC 4.1.2.15, or EC 2.5.1.54,
wherein the exogenous DAHP synthase of EC 4.1.2.15, or EC 2.5.1.54 comprises a feedback-inhibition resistant mutation in the wild-type amino acid sequence of the DAHP synthase.

24. (canceled)

25. The recombinant microorganism of claim 23, wherein
the feedback-inhibition resistant mutation is a substitution at position 180 of the wild-type amino acid sequence of DAHP synthase;
the feedback-inhibition resistant mutation is a serine to phenylalanine mutation at position 180 of the wild-type amino acid sequence of DAHP synthase; or
the wild-type amino acid sequence of DAHP synthase comprises the amino acid sequence set forth in SEQ ID NO: 1.

26.-28. (canceled)

29. The recombinant microorganism of claim 23, wherein the genetically engineered pathway:
is encoded by a single vector driven by a single promoter, wherein the promoter is:
a constitutive promoter;
an inducible promoter;
selected from a M. extorquens methanol dehydrogenase promoter (PmxaF), an E. coli (P_{BAD}) ribulokinase promoter (araBp), an E. coli (P_{lac}) β-galactosidase promoter (lacZp), or a bacteriophage lambda promoter (λP_{L}); or
encoded by a nucleic acid sequence set forth in SEQ ID NO: 6; or
comprises a nucleic acid sequence selected from SEQ ID NOs: 6, 45, 52, 53, 54, 55, 56, 57, 58, 59, 60, 61, 62, 63, 64, 65, 66, 67, or a combination of any two or more thereof.

30.-34. (canceled)

35. The recombinant microorganism of claim 23, wherein the recombinant microorganism is selected from Methylococcus capsulatus, Methylotuvimicrobia, Methylomicrobium alcaliphilum 20Z, Methylotuvimicrobium buryatense, Methylomicrobium alcaliphilum, Methylotuvimicrobium album, or Methylobacterium extorquens.

36. The recombinant microorganism of claim 23, wherein the recombinant microorganism is Methylomicrobium alcaliphilum 20Z.

37. (canceled)

38. The recombinant microorganism of claim 23, wherein the recombinant microorganism comprises a nucleic acid sequence set forth in SEQ ID NO: 45.

39.-43. (canceled)

44. The recombinant microorganism of claim 23, wherein the recombinant microorganism comprises an exogenous amino acid sequence comprising SEQ ID NOs: 1, 2, 3, 4, 5 or a combination of any two or more thereof.

45. A vector comprising a nucleic acid sequence set forth in SEQ ID NOs: 6, 45, 52, 53, 54, 55, 56, 57, 58, 59, 60, 61, 62, 63, 64, 65, 66, 67, or a combination of any two or more thereof.

46. A microorganism comprising the vector of claim 45.

47. A method of producing para-hydroxybenzoic acid (pHBA) or a derivative thereof, the method comprising:
culturing the recombinant microorganism of claim 46 in a fermentation broth;
adding a carbon source to the fermentation broth; and
isolating the pHBA from the fermentation broth.

48. The method of claim 1 further comprising at least one nucleic acid sequence encoding a polypeptide selected from:
an exogenous chorismate pyruvate lyase of EC 5.4.4.2 or EC 4.1.3.40;
an exogenous shikimate kinase of EC 2.7.1.71; or
an exogenous 3-dehydroquinate dehydratase (DHQ) of EC 4.2.1.10.
